(19) United States
(12) Patent Application Publication
(10) Pub. No.: US 2009/0043253 A1
(43) Pub. Date: Feb. 12, 2009

(54) SMART MEDICAL COMPLIANCE METHOD AND SYSTEM

(76) Inventor: Blake Podaima, Winnipeg (CA)

Correspondence Address: ADE & COMPANY INC., 2157 Henderson Highway, WINNIPEG, MB, R2G1P9, CA

(21) Appl. No.: 12/089,828

(22) PCT Filed: Oct. 11, 2006
(86) PCT No.: PCT/CA2006/001663

§ 371 Date: Jul. 29, 2008

Related U.S. Application Data
(60) Provisional application No. 60/724,882, filed on Oct. 11, 2005.
(60) Provisional application No. 60/759,070, filed on Jan. 17, 2006.
(60) Provisional application No. 60/784,798, filed on Mar. 23, 2006.
(60) Provisional application No. 60/809,362, filed on May 31, 2006.

(51) Int. Cl. A61M 5/31; H04Q 5/22

(52) U.S. Cl. 604/67; 340/10.1

(57) ABSTRACT

The smart medical compliance method and system invention prevents adverse drug events through the use of protocols that uniquely identifies the patient, care provider, medication and/or medical device that is to be used with radio frequency identification (RFID). The RFID devices incorporate fail-safe locks or indicators that prevent the inadvertent or unauthorized use of medication, medical devices, or medical supplies. The system corroborates, patient, the care provider, the medical device, and the manner in which it is to be used, and authorizes the action to be undertaken through an interface on a personal digital assistant PDA over a wireless communication channel. The system also timestamps events in the equivalent of a medical black box such that records may be kept to further improve patient care and allow an analysis of procedures. In addition, the system includes interfaces to medication preparation and safe disposal. A number of smart devices that interact with the system are also described. These include smart medical containers, smart clamps, smart valves, smart syringes, smart couplers, smart pipettes, and a host of other point of care devices.

This invention relates to a system for providing patient care at a point of care (POC).

### BACKGROUND OF INVENTION

Currently there is a heightened demand for improvements in patient point of care (POC). Errors and other incidents are inevitable in complex systems, and hence, mitigating medical errors through the use of technology and protocols via systems engineering is desirable. Over the past several years there has been increased emphasis on the reporting and analysis of POC errors. Some of the more prominent errors are erroneous patient identification, drug administration, and medication administration recording.

It is estimated that approximately 36% of adverse drug events occur at the patient POC while only 2% are intercepted [JAMA, 1995]. In addition to POC errors, there are other sources of errors including prescription, transcription, and dispensing. It is recognized that any effective system or technology for improving POC will need to be integrated within the context of a complete patient care management system.

The benefits to modernization of health management through information technology are often easily seen only once adopted. An electronic records system (ERS) introduces consistency into the process and with sufficient standards decrease errors in information gathering and processing. Practitioners like the fact that if they write a prescription, the prescription is automatically recorded. Furthermore, (personal digital assistant) PDA software can refer to the hospital or clinical system's database and list any potential interactions between the prescribed medication and other medications that the patient may already be taking.

Advancements in information and communication technology (ICT) and their adoption in healthcare necessitate a “system's approach.” Systems approaches include human factors engineering (HFE) as well as technology engineering. HFE attempts to identify situations that give rise to human errors and implement “system changes” to reduce their occurrence and minimize their impact on patients. This perspective, which strives to catch human errors before they occur, or block them from causing harm, is argued to be more effective and realizable than attempting to create an error free or flawless system. In this regard, technology engineering can be used in conjunction with HFE to improve the accuracy and efficiency of protocols and practice with a similar objective of reducing errors. Systems Engineering implies the increased use of tools such as those for failure mode and effects analysis and root cause analysis (FMEA and RCA).

There are also a number of mobile devices and wireless communication technologies that will play a major role in modernizing medical and health systems. Security is also an issue that needs to be addressed thoroughly and implemented properly to be effective as Clinical Grade Networks are developed and deployed.

“Smart” RFID devices are another technology that has the potential to improve patient safety and quality of care. Promising technologies and methodologies for improving patient POC and reducing errors include those based on barcodes and RFID. These technologies are not new and have been in commercial use for well over twenty years. They are however becoming more main-stream as both supporting electronic technology improves and connectivity protocols become standardized. One of the problems with early adoption of both RFID and barcodes is that they are inherently submissive, allowing for identification with little or no support for interactivity and automation.

Conventional applications of RFID technology in healthcare are primarily those based upon identification. These enable systems to be built around inventory tracking and control. Extensions include pharmaceutical supply chain inventory and tracking for medical reconciliation. Tied into a hospital management system, they have considerable potential to reduce adverse drug events at the patient POC. This is accomplished through corroboration of the patient ID with the drug prescribed by the physician.

In U.S. Pat. Nos. 6,139,495 issued Oct. 31, 2000; 6,032,155 issued Feb. 29, 2000 and 6,529,466 all of de la Huerga together with a number of further patents by the same Applicant is disclosed a system of controlling the supply of medication or medical events to a patient by a health care worker with the intention of reducing accidental incorrect procedures on patients.

In U.S. Pat. No. 6,897,374 issued May 24^{th} 2005, the Colder Products Company were granted priority on a connector and apparatus and method for connecting the same; however, in their invention they require an “RFID Reader” on a female end for the act or engagement of coupling. Furthermore, their device requires a hard wired connection to supplement data communications and power for actuation/control. In the smart coupler, invention described here, the mating ends require only and RFID tag 838 and associated electronics (or RFID system on a chip), as opposed to an actual RFID Reader. The control of the smart coupler invention is accomplished by way of a hand held PDA or mobile computer 115, with the actuation either being manual (human operator) or automatic (on board electromechanical latch) in nature. The design also benefits from a standardization in which both coupling ends are identical in detail. It, therefore, requires the insertion of an intermediate channel or gateway, which serves the purpose of a sterile channel to be discarded or recycled after use. (There is no intermediate channel in the embodiment of the invention described in U.S. Pat. No. 6,897,374 issued May 24^{th} 2005.)

### SUMMARY OF THE INVENTION

It is one object of the present invention to provide an improved system of this general type.

According to the invention there is provided a system for providing patient care at a point of care (POC) comprising:

an RFID tag for a care provider at the POC;

an RFID tag for a patient at the POC;

an RFID Reader;

a portable hand held computer for a care provider at the POC;

a medical device at the POC having an RFID tag;

the medical device having an operable element with a control device for enabling and disabling actuation of the operable element;

and a computing system for connecting the above items such that the control device allows actuation of the operable element only in the event that the reader detects the RFID of the care provider and of the patient and of the medical device and the computing system confirms that they are properly in accordance with a prescribed medical treatment.

The term RFID as used herein is intended to include any device which responds to an interrogation signal in a near field situation. Many different technologies are available to provide this function as mentioned hereinafter. The device may be incorporated with elements effecting other functions such as wi-fi communications.

Preferably the computing system is arranged to provide a time stamp record of an actuation of the operable element.

Preferably the medical device includes a sensor for detecting operation and a completion of an operation and wherein the computing system is operable to record both operation and completion.

Preferably the computer system is arranged to provide a reminder to the portable hand held computer if not completed.

Preferably the computer system is arranged to provide messages to the portable hand held computer providing a control of workflow for the care provider.

Preferably the computer system is arranged to provide a message to the portable hand held computer of a second care provider in the event that the first care provider provides an indication of an inability to complete a workflow task.

Preferably there is provided a manual override key which can be engaged with the medical device for overriding the control device.

Preferably there is provided a series of medical devices with common interface for driving actuation of said operable element and a module separate from the medical devices including a battery, drive member and control device for operating the series of medical devices.

Preferably the module includes a reader for reading a tag on each of the medical devices and wherein the computer system is arranged to allow operation thereof only in the event that the correct medical device is connected.

Preferably the RFID tags and the computer system include security protocols.

Preferably the RFID tags and the control device are programmable and reusable.

Preferably the RFID tags and the control device are arranged to tolerate temperature, chemical, and/or electronic processes.

Preferably the computer system is arranged to prevent operation of the operable element if the medical device is not sterilized.

Preferably the computer system is arranged to prevent operation of the operable element if it is beyond an expiry date.

Preferably the computer system is arranged to provide on the portable hand held computer details of allowable use of the medical device.

Preferably the RFID tags provide Remote coupling (0-1 m).

Preferably the RFID tags and the computer system include protocols for Data integrity.

Preferably the reader reads multiple RFID tags by a protocol utilizing a windowed access mechanism of a plurality of slots, with a series of transponders contending for a slotted channel in a random access fashion.

Preferably the medical device comprises one of smart containers, smart clamps, smart valves, smart couplers, smart syringes, smart pipettes, smart bandages and smart catheters.

Specific details of these devices is provided hereinafter and each of these devices may include features which are independently patentable.

Preferably the medical device includes a “tamper-proof” or “breach” indicator.

Preferably the medical device includes a visual aid providing information to the care provider.

Preferably the portable hand held computer has at least a part of the electronics thereof juxtaposed with the RFID Reader. So that the RFID tag of the care provider is part of the Hand held computer. Or the care provider may have a separate RFID for ensuring authorized use of the Hand held computer.

Preferably the medical device at the POC has an RFID tag juxtaposed with interfacing electronics forming at least part of the control device (perhaps RFID System on a Chip).

Preferably the control device is arranged to disable operation of the operable element. Although as an alternative it may merely provide visual or other indication to the care provider that the computer system indicates that the operation is proper so that the care provide may proceed.

### BRIEF DESCRIPTION OF THE DRAWINGS

Embodiment of the invention will now be described in conjunction with the accompanying drawings in which:

FIG. 1A is a schematic illustration of a Medical Compliance System according to the present invention.

FIG. 1B is a schematic illustration of a Medical Compliance ICT System according to the present invention.

FIGS. 2A, 2B and 2C together provide a schematic illustration of a Screw Clamp (slide on and hinged type: mechanical instance) according to the present invention.

FIGS. 3A, 3B and 3C together provide a schematic illustration of a Screw Clamp (slide on and hinged type: electromechanical instance) according to the present invention according to the present invention.

FIGS. 4A and 4B together provide a schematic illustration of a Cam Clamp (mechanical instance) according to the present invention.

FIGS. 5A and 5B together provide a schematic illustration of a Cam Clamp (electromechanical instance) according to the present invention.

FIGS. 6A and 6B together provide a schematic illustration of a Scissor Clamp (mechanical instance) according to the present invention.

FIGS. 7A and 7B together provide a schematic illustration of a Rotational Clamp (in-line or clam shell type: mechanical instance) according to the present invention.

FIG. 8 is a schematic illustration of a Rotational Clamp (in-line or clam shell type: electromechanical instance) according to the present invention.

FIG. 9 is a schematic illustration of a Push-type Clamp (in-line or clam shell type: mechanical instance) according to the present invention.

FIG. 10 is a schematic illustration of a Lever-type Clamp (in-line or clam shell type: mechanical instance) according to the present invention.

FIG. 11 is a schematic illustration of a In-line Latch Clamp: (mechanical instance) according to the present invention.

FIGS. 12A and 12B together provide a schematic illustration of a schematic illustration of a Hinge Clamp (mechanical instance) according to the present invention.

FIGS. 13A, 13B and 13C together provide a schematic illustration of a Linear-Actuator Ram Clamp (mechanical instance) according to the present invention.

FIGS. 14A, 14B and 14C together provide a schematic illustration of a Linear-Actuator Ram Clamp (electromechanical instance) according to the present invention.

FIGS. 15A, 15B and 15C together provide a schematic illustration of a Roller-Actuator Clamp (mechanical instance) according to the present invention.

FIGS. 16A, 16B and 16C together provide a schematic illustration of a Roller-Actuator Clamp (electromechanical instance) according to the present invention.

FIGS. 17A, 17B, 18A and 18B together provide a schematic illustration of a Stop-cock [Cylinder] Valve (mechanical and Electromechanical instance—2 Port and 3 Port, respectively) according to the present invention.

FIGS. 19A and 19B together provide a schematic illustration of a Stop-cock [Cylinder] Valve (mechanical and Electromechanical instance—2 Port according to the present invention.

FIGS. 20A, 20B, 20C and 20D together provide a schematic illustration of a Stop-cock [Cylinder] Valve (mechanical and Electromechanical instance—2 Port 4-way) according to the present invention.

FIGS. 21A to 21E together provide a schematic illustration of a Butterfly Valve (mechanical and electromechanical instance) according to the present invention.

FIG. 22 is a schematic illustration of a Gate, Globe, needle Valve (adjustable screw—mechanical instance) according to the present invention.

FIG. 23 is a schematic illustration of a Gate, Globe, needle Valve (adjustable screw electromechanical instance) according to the present invention.

FIGS. 24A and 24B together provide a schematic illustration of a Syringe and RFID with Control Mechanism at Nozzle according to the present invention.

FIGS. 25A, 25B and 25C together provide a schematic illustration of a Syringe which is Fail-safe RFID with Control Mechanism at Finger-Flange according to the present invention.

FIG. 26 is a schematic illustration of a Syringe which is Operator Responsible—RFID with Indicator Only according to the present invention.

FIGS. 27A, 27B and 27C together provide a schematic illustration of a Syringe which is Fail-safe RFID with Rotation and Push-pull Latch Mechanism according to the present invention.

FIGS. 28A and 28B together provide a schematic illustration of a Syringe which is fail-safe RFID with Finger-Flange Module Assembly according to the present invention.

FIGS. 29A and 29B together provide a schematic illustration of a Syringe which is fail-safe—RFID with Control for Legacy Syringes according to the present invention.

FIGS. 30A and 30B together provide a schematic illustration of a Syringe with RFID with Collapsible Latch Mechanism according to the present invention.

FIGS. 31A to 31D together provide a schematic illustration of a Syringe with Possible Position (Resolver) Sensors according to the present invention.

FIGS. 32A and 32B together provide a schematic illustration of a Syringe with Possible Removable Thumb-rest Implementations according to the present invention.

FIG. 33 is a schematic illustration of a Syringe with Fail-safe-RFID with Intersticed control device according to the present invention.

FIGS. 34A and 34B together provide a schematic illustration of a Syringe with RFID with Motorized Control and Actuator Device according to the present invention.

FIGS. 35A and 35B together provide a schematic illustration of a Syringe with Fail-safe—Alternative Implementation (Cylindrical Plunger) according to the present invention.

FIG. 36 is a schematic illustration of a Universal Smart Key according to the present invention.

FIG. 37 is a schematic illustration of a Coupler (MFM configuration shown, FMF similar) according to the present invention.

FIG. 38 is a schematic illustration of a Smart Pipette according to the present invention.

### DETAILED DESCRIPTION

This invention presents RFID technology within a medical context and introduces novel designs using enhanced RFID devices (system and methodology) for integration within evolving and legacy POC systems. It provides a conceptual overview of the point of care interacting components within the medical reconciliation and compliance platform. A smart medical device and its system of deployment include methods of identification and control for medical compliance. Identification is accomplished with the aid of RFID, while control is enabled through a mechanism that can be activated to prevent improper or unauthorized access.

Smart RFID devices attempt to facilitate error-free dispensing and administration (of medication and/or medical supplies), and other clinical practices, to reduce or prevent adverse medical events, near misses, or sentinel events. These devices may incorporate an RFID enabled electromechanical lock or latch controlling their access and include smart medical containers, smart clamps, smart valves, smart syringes and pipettes, smart IV pumps, smart couplers, and smart bandages. The RFID tags on these devices can be either active or passive, and the control and communication can be derived from the interaction of an RFID reader and tag in conjunction with the associated electronics and overseeing medical information management system.

RFID enabled devices come with an associated overhead, but are not superfluous in deployment, and can be used within the framework of an engineered POC system. The designs disclosed herein offer seamless integration with purposeful function, and an evolutionary path to improved overall medical compliance. Future RFID devices will extend beyond traditional uses—even the “smart” applications disclosed here. Such RFID devices will incorporate various sensors and will be widely available as implantable devices.

RFID technology utilization is gaining momentum and is being tailored to a number of applications. Although there are a variety of RFID tags and systems, those best suited to health care have a number of differentiating characteristics. More specifically, an RFID transponder or tag in a medical application will require data capacities that range from a few bytes to several kilobytes. In contrast there are 1-bit transponders which provide information only on their presence. Although inexpensive, and likely to be widely applied in commercial environments, they are less likely to find much utility in a health setting.

RFID transponders that allow for sufficient data require an integrated circuit and have more stringent power requirements. This power can be derived through an interrogating electromagnetic field of a reader, or supplied by an on-board battery. Typically, an RFID transponder will interact with a reader in one of two ways: either simultaneously interacting (with a reader) over a modulated channel, or in a sequential manner, where the reader switches off the interrogating field allowing for the transponder on the tag to respond during a quiescent period.

In addition to requiring data storage, a health related RFID system will also require security beyond that found in many commercial applications. Security protocols and their processing imply an additional constraint upon the energy requirements of the RFID device itself. Many medical RFID devices will also be required to interact with a sensor, activate a solenoid or motor (or other electromechanical device) thereby increasing the power requirements still further.

Medical RFID devices could also be programmable and reusable. The reuse implies an additional constraint that may require the device to be subject to temperature, chemical, and/or electronic processes, not otherwise needed in less sterile environments. As with other medical devices, clinical grade medical RFID devices will be required to meet the stringent standards of various governing bodies and institutions of the health industry. Clinical grade RFID devices will also be required to meet rigorous EMI and EMC (electromagnetic interference and compatibility) guidelines.

The frequency of operation for RFID devices fall into several broad ranges reflecting that of the reader. These range from RF (MHz) to microwave (GHz). The physical operating proximity of devices is also an important issue in a medical setting. It is likely that close coupling (<1 cm) would contravene the existing protocol of a POC practitioner. Remote coupling (0-1 m) would allow for the functionality of the RFID device without compromising the protocol of the practitioner or care provider who may be wearing the reader/transceiver on his or her wrist or belt.

The basic operation of an inductively coupled 13.56 MHz (ISM band) RFID transponder (tag) is as follows. The transponder couples with the RF field of the reader. In this case the reader is operating at 13.56 MHz. The transponder is tuned to this frequency (powered by the ambient field of the reader) and modulates a sub-carrier with the code (ID) stored on the transponder. This code effectively load-modulates the impedance seen by the reader at sideband frequencies on the order of +/−424 KHz. This provides a sideband that is filtered by the transceiver of the reader and demodulated to determine the ID of the transponder. Variations on this basic idea include alternative coding or keying as well as modulation methods. Data integrity is a crucial aspect of an RFID system in a medical application. At the lowest level the most effective and efficient error control check is that provided by a Cyclic Redundancy Check (CRC). In theory a CRC provides error aliasing performance on the order of one part in 2ⁿ, where n is degree of the CRC polynomial, or equivalently, the number of bits associated with the CRC register. A CRC is easily implemented in minimal hardware consisting of D type Flip-flops and a small number of exclusive-or gates. In RFID operation the transponder transmits its data (e.g., ID and sensor data) and a CRC is calculated within the transponder and this value appended to the transmitted block of data. The reader calculates the CRC on the received data, (e.g., ID, sensor data, and appended CRC). If this CRC is zero (easily checked in hardware) the received data is assumed to be error free. One precautionary note is that the aliasing behavior of an n-bit CRC being 1 in 2n is typically an asymptotic result, and hence, an overestimate of the actual error performance. The relatively limited size of medical RFID data actually places the aliasing performance of the CRC in its transient analysis domain. As such, further analysis of the CRC and its behavior should be undertaken if it is to be used in critical medical RFID environments.

If the aliasing behavior of the CRC is not sufficient, simple additional error control, such as redundant reading or polling, can easily be implemented without having to resort to stronger error control techniques.

An incorrect CRC is an indication of at least one bit in error resulting from interference, or a weak signal to noise ratio. If one is interested in securing the data in a manner ensuring integrity and authenticity, a public key encryption standard such as RSA can be implemented. With strong encryption, however, there is a computational requirement that may be difficult to budget for on an extremely low power device. Public key encryption offers easier key management than secret key systems, but at the expense of having higher computational requirements. If hardware efficiency and security are required, a public key system can be used to exchange a secret key that can be implemented in a streaming cipher, not significantly more complicated than the CRC physical layer protection, as previously discussed. Issues such as renewal of the secret key will have to be taken into account if one is to guard against simple replay attacks or forgeries. Fortunately, many of these techniques are being addressed within the wireless LAN community and can be modified or redeployed within an RFID environment.

In addition to issues associated with general RFID operation (error control and security) multiple-access within a medical environment requires consideration. It is envisioned that with many medical applications a reader may be in close proximity to a number of RFID transponders. The problem of multiple-access within a shared medium has been encountered and addressed in wired technologies such as 802.3 (Ethernet) as well as wireless technologies such as 802.11x. A difficulty with respect to wireless technologies, in general, is that if a transponder is broadcasting it can not hear other transponders that may also be broadcasting—making it basically a free-for-all with collisions severely limiting throughput. However, it should be noted that the efficiency of an RFID system may not be as adversely affected as other radio systems for the following two reasons: one, the number of transponders in the spatial vicinity of an interrogating reader is anticipated to be relatively small; and, two, the amount of data is also relatively small. For instance, a protocol utilizing a simple windowed access mechanism of 16 slots, with 5 transponders, contending for a slotted channel in a random access fashion (supporting a data rate on the order of 25 Kbits/sec, and an average read time of 30 msec,) the probability of successful packet reception would be 77%. As such, within a short period (less than 3 windows, 1.5 sec.) all 5 RFID transponders would be read with high probability (0.99). If this simple scheme were not sufficient, a reader could poll individual RFID transponders in a similar manner to other radio contention resolution schemes. This of course requires that the transponder be provisioned with sufficient electronics to respond when queried—as an individual device or within a group or transponders. In either case, it should be noted that collision avoidance may be an issue in a medical setting and requires proper engineering consideration.

### Smart Medical Compliance System and Platform

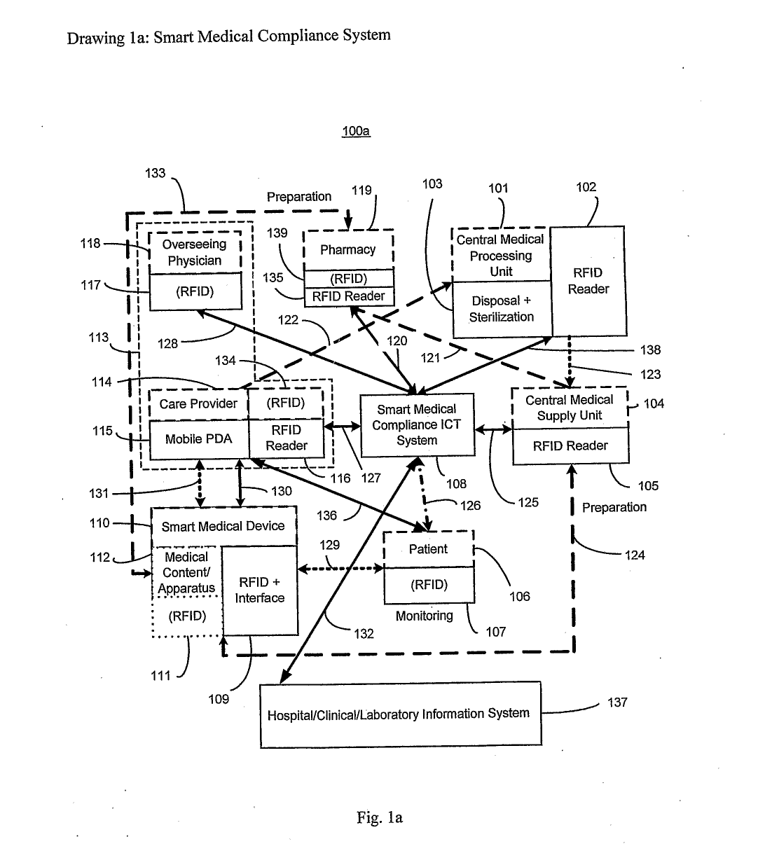

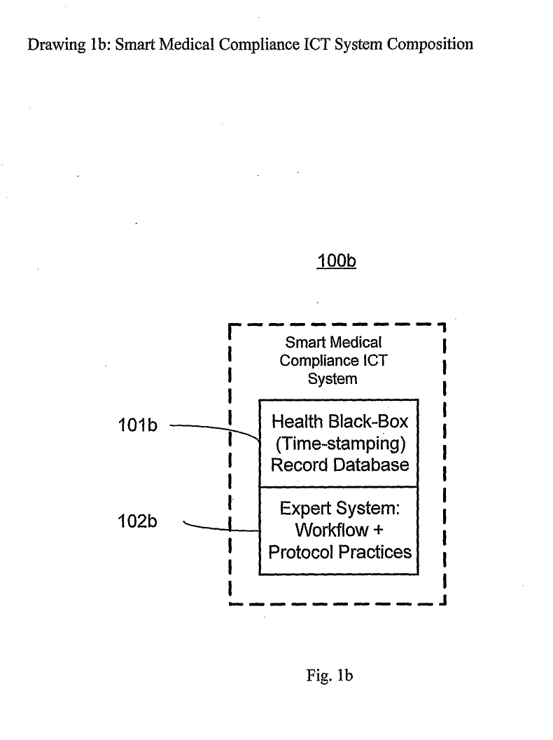

FIG. 1a is a diagram illustrating a system 100a for the smart medical compliance system, and interacting medical components, as an example of the embodiment of the invention. The system 100a may be implemented in a healthcare facility, hospital, personal care home, clinic, laboratory, etc., wherever there is an existing and supporting information and communication technology (ICT) infrastructure. The conventional or legacy ICT infrastructure (and the connectivity of the facility), from a Systems Engineering perspective, is not shown (for simplification) in the illustration of FIG. 1a. That is, existing interfaces and communication channels are omitted in the System's view of the smart medical compliance system for purposes of clarity only. It should be understood that some of these channels are available to the smart medical compliance system and may indeed be shared. Some of the departments or entities which may have their own established independent communications channels (outside of the smart medical compliance system and its middleware) are the care provider or clinical technician 114, the overseeing physician 118, the pharmacy 119, the central medical processing unit 101, the central supply unit 104, and the patient 106.

The following defined communication links (in some instances) may be understood as being visual, auditory (as in verbal communication), or gesturing (as in sign-language), (as in verbal communication) in nature, or actual hard wired or wireless data/information communication channels. The default implication is that the links are either hard wired or wireless data communication channels. The visual or auditory channels of communication will be mentioned or described explicitly in the correct context so as to eliminate any possibility of confusion. Depending on the kind of smart medical device/apparatus and its communication requirements (i.e., medium or long range wireless, or near field communications, NFC), wireless communication may be ubiquitous in or outside the facility as long as there is an active communication channel available via wireless access points. It should be noted that, as illustrated in FIG. 1a, RFID tags are denoted as “(RFID)” as in the overseeing physician's RFID tag 117, the pharmacist's RFID tag 139, the patient's RFID tag 107, and the smart medical device's RFID tag 111. On the other hand, an RFID reader is denoted as “RFID Reader,” as in the pharmacist's RFID Reader 135, the central processing unit's RFID Reader 102, the central medical supply unit's RFID Reader 105, the care provider's RFID Reader 116, and the smart medical devices RFID Reader (not shown, but there is an option to add one for certain devices, or in certain circumstances—as in the deployment of say a smart medical container).

The smart medical compliance ICT (information and communication technology) system 108 is interfaced to a hospital and/or clinical and/or laboratory information system 137 via communication link 132. The smart medical compliance ICT system 108 is interfaced to persons such as the patient 106 (monitoring), the care provider or qualified worker (or clinician, or technician) 114, and/or the overseeing physician 118 (can be generally referred to as the primary care providers 113) and/or pharmacist 119. These persons are identified by their RFID tags thereby identifying the patient 107, the care provider 134, and/or the overseeing physician 117 and/or pharmacist 139. The care provider and/or overseeing physician 118 also has a mobile Personal Digital Assistant (PDA) or handheld computer 115 and RFID reader 116. Although it is not explicitly shown, the overseeing physician can also have a mobile PDA or hand held device. (This fact is covered in the illustration of FIG. 1a by noting that the overseeing physician and the care provider, clinician, or technician, may be one in the same person, i.e., a “general” health care provider 133.) The smart medical compliance ICT system 108 can communicate with the patient 106 via communication link 126 (in the event that monitoring is required—for instance, as in intensive care vital sign monitoring). Furthermore, the smart medical compliance ICT system 108 can also communicate with the care provider 114 via communication link 127 and with the overseeing physician 118 via communication link 128.

The smart medical compliance ICT system 108 is interfaced to departments such as pharmacy 119, central medical supply unit 104, and the central medical processing unit 101. Pharmacy 119 is equipped with an RFID reader 135, and can communicate with the smart medical compliance ICT system 108 via communication link 120. The central medical processing unit 101 is equipped with an RFID reader 102, and can communicate with the smart medical compliance ICT system 108 via communication link 138. The central medical processing unit 101 is also equipped with a disposal, sterilization (and reconstitution) unit 103. The central medical supply unit 104 is equipped with an RFID reader 105, and can communicate with the smart medical compliance ICT system 108 via communication link 125. Pharmacy 119 (or the pharmacist, RFID tag 139) can communicate with the central medical supply unit 104 via communication link 121. The central medical processing unit 101 can communicate with the central medical supply unit 104 via communication link 123.

An example of a smart medical device is represented by 110. These include, but are not exclusive to, smart containers, smart clamps, smart valves, smart syringes, smart pipettes, smart bandages, smart catheters, and a plethora of smart surgical tools, devices, and apparatuses. The smart medical device 110 includes an RFID tag 111, medical content and/or apparatus 112, RFID and associated interface (such as RFID system on a chip and other electronics and computing hardware) 109. The smart medical device 110 can communicate with the care provider 114 and/or physician 118 (visually or audibly) or via communication links 130 and 131. The smart medical device 110 can also communicate with the pharmacy 119 via communication link 133 (at the point of preparation, at the patient point of care, in surgery, or even anywhere within the facility as long as there is a communication channel available). The smart medical device 110 can communicate with the patient directly (visually or audibly) or the patient's RFID 107 via communication link 129, and with the central medical supply unit 104 via communication link 124. It should be noted that depending on the kind of smart medical device/apparatus to be deployed, its point of preparation may be in Pharmacy or the central medical processing lab, or both, at which time the smart medical device/apparatus can be programmed and prepared for the deployment within the facility or for home use on an out patient basis.

Pertaining to a more human factors involvement in the “chain of command” of facility operations, the care provider 114 can communicate with the central medical processing unit via communication link 122. Moreover, the care provider 114 can also communicate with the patient 106 verbally, or with sign-language, and/or via communication link 136. This data or signal communication path is established using the RFID Reader 116 and mobile PDA 115 for the reading and/or (re-)programming the RFID tag 107 (wristband tag) on the patient. This may simply be for the purpose of positive identification (for corroboration), prior to the commencement of a medical procedure, so that a patient does not receive an incorrect or unassigned medical treatment leading up to an adverse event, near miss, or sentinel event. This may also be for the purpose of updating (or uploading) or downloading the patient's “on-board” tag's point of care (or surgery) record/history, or other medial records. In this way, it is possible that every administration, procedure, or service can be logged/transmitted not only to the main information management system and data base via the mobile PDA 115, but to the RFID tag 107 itself where it will reside in memory to be polled or interrogated at a later time perhaps even by other departments. This can be a useful feature (for department personnel) in determining the status of a patient when he/she is transferred from department to department for various medical testing/tests.

### Health Information Technology Management System

The health IT system 137 is system is responsible for the entire health information and communication technology (ICT) for an entire healthcare and/or laboratory facility. This system is comprised of several components. However, at the heart of the system is the electronic medical records and medical administration records system which includes patient information data bases, and storage systems for medical imaging (CAT, MRI, ultrasound, etc.) and medical or laboratory tests. Other services such as billing, accounting, inventory, payroll, and human resources, may be performed as well.

The “middleware” of the described smart medical compliance system 100a interfaces the main medical information system (or the hospital information technology IT system 137) with a smart medical compliance ICT management system 108. This includes a heath black box (time stamping) record database 101b and an “expert system” 102b responsible for workflow, protocol and practices expert system. Its deployment is primarily for the safe and efficient management of heath care procedures, services, and personnel, at the patient point of care and surgery, in facilities such as hospitals, clinics, laboratories, and personal care homes.

### Pharmacological Preparation and Dispensing (Pharmacy)

An instance of medical compliance is realized at the pharmacological preparation and preparation/dispensing point. In this scenario, the pharmacist 119 is required to correctly identify himself via RFID tag 135 and fill a prescription with the appropriate medication or supply, etc., for the corresponding patient 106—as well as identify the care provider 113 who is responsible for the actual act of prescription (overseeing physician 118) and administration (care provider 114). It should be noted that the pharmacist would also be responsible for the correct preparation of smart medical devices/apparatuses 110 (and its contents, in the case of a smart medical container) to be handed off ultimately to the care provider 114 to perform the actual administration of the medical content (i.e., medication, preparations, supplies).

Central Medical Processing and Supply Units: Recycling—Disposal. Sterilization, Reuse, and Distribution

The Smart medical device/apparatus operational components may require sterilization for safe reusability. Proper handling and disposal protocol (i.e., Bio-hazard compliance) may require that an RFID Disposal Reader log the RFID tag of the smart medical device/apparatus and time-stamp the disposal, sending this information back to the main information management or records system. Even the reconstitution of the smart medical device/apparatus for re-use may require time-stamping in its preparation prior to redistribution. Furthermore, the smart medical device/apparatus can be “programmed” or initialized to identify itself and/or its content and components for proper disposal, re-sterilization, reconstitution, or recycling. A secure tamper-proof inlay indicator may be affixed (if required) at this point of preparation.

Moreover, the central medical processing 101 and central medical supply 104 facilities can also perform a vital service in the supply and distribution of smart medical devices/apparatuses. Using RFID Readers in conjunction with the smart medical devices (and perhaps any or all of their contents/components, which may also be “RFID tagged”) at various stages of disposal, recycling, or reconditioning, the process of reading (downloading) or reprogramming/resetting of their RFID tags (or RFID systems on a chip) can reveal critical information related to issues of supply and inventory management. This acquired information can be used in the operations management of the facility. For instance, knowledge related to utilization and duty cycle (or the number of times the devices have been placed in service) can be used by the central medical supply unit 104 in the planning of inventory or storage (including the distribution, location, and co-location of stock). The numbers of smart medical devices/apparatuses in the field and their identification can also be readily obtained. When this is combined with tracking and location detection systems (also using RFID or other similar identification and location technology) a plethora of useful or even critical information can be made available to managers and other authorized personnel in the facility.

### Medical Content Disposal

After administering medical content or performing a medical procedure or service via a smart medical device—at the time of proper disposal—the RFID tag of any residual medical content (or expended medical devices) could be re-read and the event time-stamped. An RFID reader located on a disposal chute or disposal apparatus could read the RFID tag of the expended medical content, time-stamp the event, and send this information back to a central main medical information management or patient medical records data base system. Hence, this information can be used to “close the loop” on when and where the medical content was administered providing an ancillary level of medical compliance. The reading of the RFID tag of the expended medical content and/or smart medical device/apparatus at time of disposal is useful even if the RFID enabled latchable/lockable device or its contents or components were not used. The disposal RFID reader would simply log the identification of the RFID tag of the smart medical device/apparatus, its components, or its residual medical content, and time-stamp the event sending this confirmation message or signal back to the main medical information or patient medical records data base system.

### Security (Tamper-Proof Inlay) Indicator

A security “trip-wire” (conductive strip/trace, decal, inlay, or pin, security seal) can be incorporated into a smart medical device/apparatus affixed or attached by adhesive or glue (or other fastening means, using for example, screws or pins) as a “tamper-proof” or “breach” indicator. This passive or electrically conductive inlay strip or decal is placed over the latch/lock, or attached to a movable (pivoting/sliding/rotating) structure and housing embodiment, which precludes access to the gate/door or latch, or the operation therein of the smart medical device, respectively, only to be removed by authorized personnel with proper protocol and practice. The removal would entail the eradication of a tab on the security seal, or otherwise of some portion thereof, and in doing so activating the “compromised” state of the smart medical device/apparatus—indicating that a breach has occurred in the process. One method of accomplishing this is with the use of a simple visual indicator in the form of an inlay or decal made or designed to be obvious to an operator (or others) in determining if it is breached, destroyed, or tampered with. On the other hand, the security seal of a smart medical device/apparatus can also be electrically or mechanically connected to dedicated smart medical device/apparatus alarm circuitry. It can also be attached or affixed to an entirely separate RFID inlay or the already residing RFID electrical circuitry itself (or RFID system on a chip) responsible for the control or actuation of higher order smart medical device/apparatus functionality. If the inlay, strip, or pin is electrically coupled in either of these manners, an alert signal can be activated in event that the “sealed” or securely prepared smart medical device/apparatus has been inadvertently or deliberately opened or tamped with. This is an incorporated security feature to ensure that the medical device/apparatus and its associated contents have not been tampered with. Indication of a broken or tampered seal can be revealed on the smart medical device/apparatus itself either visually, audibly, or both, through dedicated alarm electrical circuitry. It can also be manifested by way of a “state-change” through the onboard RFID electrical circuitry (which can be polled or interrogated by the RFID reader) whose status can be indicated on the display screen and/or speaker of a hand-held computer, PDA, or mobile device/cart, to be relayed to an overseeing information management system. In this way, a visual (e.g., flashing Red light) or unique audio alarm indicator or can be incorporated in the smart medical device/apparatus itself (and/or a separate supporting monitoring device) as an indication of content integrity. This will help ensure security, proper compliance, and administration integrity in a medical setting. In the event that the integrity of a smart medical device/apparatus has been compromised, the overseeing medical management system can make a determination regarding clinical or laboratory security protocol and practices to alert those authorized parties in charge or responsible for circumventing any potential wrongdoings or criminal behaviors

### Smart Medical Compliance ICT System

FIG. 1b is a diagram of the smart medical compliance information and communication (ICT) system 100b which illustrates the smart medical compliance information and communication (ICT) system or component 108 of the overall smart medical compliance system or platform 100a of FIG. 1a. The smart medical compliance information and communication (ICT) system depicted in 100b is comprised of components (or sub-components). These components include a health black-box 101b (providing time stamping) with record data base, and an (intelligent, or simple) expert system 102b managing operations related to personnel work flow, protocols and practice, of and within the facility.

### Health Black-Box (Time Stamping)

The health black box 101b is a subsystem that records information related to medical and/or clinical practices in laboratories and/or at the point of care, including surgery (using mobile PDAs or hand held computers, in conjunction with smart medical devices/apparatuses), at testing and treatment locations (using a variety of computer and communications services and technologies that may be available, such as, desktop computers, mobile carts with computers, mobile PDAs or handheld computers, or computer interfaces on the actual testing or treatment machines themselves)—wherever there may be a care provider, practitioner, clinician, or technician with an RFID Reader who is part of the overall smart medical compliance infrastructure. The information recorded by such a system may only be accessible to authorized personnel, such as, operations managers, safety and quality of care professionals and executives, and policy makers within the facility, or even of a higher authority (standards bodies, or government), with the responsibility of improving, maintaining, and assuring a certain level or standard of healthcare practice. This system can if so desired be programmed to automatically alert authorized and assigned personnel of any breach or collapse in policy or practices, and/or any medical errors (adverse events, near misses, or sentinel events) than may have occurred requiring attention for immediate intervention or improvement, or for sometime (according to set protocol and practices) at a later date/time.

### Expert System (Workflow, Protocol, and Practice Manager)

The expert system 102b is responsible for personnel workflow, protocols, and practices, in and within the facility. By definition, it is an automated system for performing logical deductions and inferences from a set of known facts which are embedded in knowledge based rules. This system is also capable of making new inferences (without intervention) on new facts by exercising these rules via software running on a computer as part of the smart medical compliance ICT system 108, 100b. Information is propagated through communications channels to and from receiving and transmitting devices (e.g., mobile computers, and smart medical devices/apparatuses) comprising a significant component of the overall smart medical compliance system 100a. As it relates to the smart medical compliance system and method, these rules form the basis of logically managing the clinical practices of working personnel. The “appropriate” or “desirable” working practices and conditions can be inserted or programmed in the system, for instance, by those skilled in systems (human factors) engineering and operations management, in such a manner, as to make for safe and efficient medical delivery, practices, and standards within the facility.

Smart medical devices/apparatuses 110 can be “enabled” or “disabled” according to such a set policy, for the main goal of significantly reducing medical errors, to in turn, improve safety, efficiency, and quality of care. Of course, along with achieving reduced errors are the several cost saving benefits that can be realized: less patient hospitalization time, fewer law suits (litigation cases), less re-testing, and more efficient uses of resources. There are also the costs savings that are attributed to avoiding a bad reputation (which is extremely important as healthcare facilities are constantly being rated and scrutinized) to facilities which are at less risk for incurring significant medical errors. It should be also noted that there are efficiencies to be accrued by having working staff members operate in a timely and efficient manner. They may also increase their productivity if their work is less stressful and more enjoyable, yielding to less sick leaves or injuries on the job—and leading to further cost saving benefits.

This expert system is also responsible in handling workflow in the event of adverse or extenuating circumstances. For example, if a care provider is called to an emergency, he/she can enter a “state” or code via their PDA to indicate that were “called away” to a more pressing medical issue. The expert system, after being made aware of the pending emergency and “sign-off” of the care provider, will “call in” (notify) a substitute worker (through their PDA) to perform the previously assigned/pending medical task in a seamless fashion. If on the other hand, the medical task is not performed, an open “loop” will be recognized, and another option may be exercised, depending on the policy programmed into the expert system (such as warning delivered to one's PDA, or to a higher authority). A hierarchy is embedded in the expert system designed for assigning and reassigning work flow duties to find qualified and available personnel. Also embedded in these rules are protocols at the point of care in the event of device failure, whereby replacement procedures are governed by the expert system. The same is can is true for re-staffing (due to shift changes and holidays).

### Ubiquitous or Pervasive Health Computing Environment

The described smart medical compliance, method and system (and its smart medical devices/apparatuses 110) can be envisaged to subsist within a ubiquitous or pervasive health computing environment. In this manner, small embedded computers (mobile PDAs, or hand held computers 115) would respond to one's presence, desires, and needs, without the operator necessarily being solely responsible for all active manipulation within one's environment. This can be accomplished with the benefit of a health expert system 102b. A network of fixed and mobile wireless devices would allow for communications so as to seamlessly integrate the operator's intentions and even perform tasks automatically. This will rid the operator of the more error-prone, mundane, and arduous tasks, freeing up time necessary to focus on the primary task at hand. In this manner, their work and other unexpectedly assigned activities should be made easier (and perhaps even more enjoyable) while their presence is more transparent, making for a less intrusive and invasive practice.

### Smart Medical Compliance: Operation and Protocol

It is envisaged that the smart medical devices/apparatuses 110 operator (i.e., care provider 113) at the point of dissemination would have an RFID tag 134 and RFID Reader 116, with a personal digital assistant (PDA) 115 or other portable or mobile wired/wireless hand held computer (integrated or stand alone). In this instance, a mobile computer (cart) or PDA is capable of communicating with a smart medical device/apparatus 110 and concurrently capable of communicating with the main information management or records system. This later communication could be provided through a wireless or wired network, intranet, Internet, cellular or telephone system. As previously mentioned, with respect to the hand held computer 115, the RFID Reader 116 could be attached, built in, or detachable, with wired or wireless communication or stand alone capability (as is the case with a universal smart key, described below).

The protocol of operation calls for the operator to interrogate the RFID tag (wristband tag) of the patient 107 and of the smart medical device/apparatus 110, with the handheld PDA 115 RFID Reader 116, and upon corroboration (matching the correct identity of the patient 106 and the smart medical device/apparatus 110 with the desired actuation state) the smart device/apparatus 110 could be electromechanically unlocked (or unlatched) and the smart medical device/apparatus mechanism enabled or prepared for actuation. (The process of authentication can also include a biometric interface (for patient 106 and care provider identification 114, or 118, to further corroborate operator access, permissions, and authority.)

The process described above can also provide services including time-stamps, sensor information acquisition, and operational data collection—that may optionally be logged back to the health black box record database 101b of the smart medical ICT system 101b or even the main health information management records system through this gateway.

A “failsafe protocol” can also be affected by the expert system 102b within the scope of the present smart medical compliant method and system 100a. In the event of smart medical device/apparatus 110 failure, the operator (care provider 114 or qualified technician) has prior knowledge and procedures for overriding and replacing the malfunctioning smart medical devices/apparatuses 110 without compromising intended purpose, operation, and functionality. The smart medical device/apparatus performs “self test diagnostics” periodically, upon power up, shut down, or query. In this manner, any anomaly will be reported to the care provider or qualified and authorized technicians for override, repair, or intervention. The onboard intelligence of the smart medical device/apparatus itself will provide an audio or visual warning, or through wireless communication, provide a warning to the screen of the hand held PDA 115 or mobile computer, notifying the operator or qualified technician of a hardware failure (or pending failure). The operator may now invoke an override procedure to manually bypass the failure and continue with intended smart medial device/apparatus function in compliance with failsafe protocol. The overall operation is not compromised since the failure mode has been recognized and recorded, thus allowing steps to be undertaken to repair or replace the defective smart device/apparatus in a controlled and monitored manner. This process is overseen by the smart medical compliance ICT management system 108 to conform to standards for minimizing the presence of a defective smart medical device/apparatus in the field. The compliance conforms to requirements within each field of application and deployment. The purpose is to safeguard against erroneous, unauthorized, malicious, or inadvertent (accidental) use, and moderate the handling, management, or replacement, of defective or obsolete devices. The actual override procedure is performed by an “authorized operator” or other authorized personnel (qualified technicians) utilizing a manual override procedure. The override personnel should have the necessary authority to reconstitute a replacement smart medical device/apparatus to a new or restored state or operational state. Any and all information gathered from the override procedure and replacement procedure would be accessible via the smart medical compliance ICT management system. All available information gathered via the mobile PDA, handheld, or cart computer can be communicated to the smart medical compliance ICT management system for real time feedback/notification and/or post incident investigation and analysis to a third party (hospital authorities, standards bodies, or policy makers).

### Smart Medical Therapy and Compliance

At present a non-negligible number of medical incidences (adverse events, sentinel events, and near misses) occur comprising of errors and accidental and/or incorrect drug administrations to patients—as a direct result of ineffective identification and control practices and protocols. The invention described herein therefore relates to medication compliance at the patient 106/care-provider 114 interface (patient point of care, surgery, treatment, or testing), transport, supply, distribution 104, reconstitution 101, and/or pharmacological preparation and dispensing point 119. The device, method of deployment, and management system, addresses both identification and control. The identification is accomplished with the aid of Radio Frequency Identification (RFID), while the control is provided through a mechanism that can be activated or deactivated (via RFID and associated electronics, and/or RFID System on chip technology) to prevent improper, erroneous, accidental, or unauthorized access, or to facilitate error-free preparation, dispensing, transportation/delivery, and administration of a medical preparation, service, or therapy.

In one instance, at the patient point of care, the patient 106 and care provider 114 would be identified by their RFID tags 107, and 134, respectively, and the smart medical device/apparatus by its RFID tag 111. These devices would be affixed to the patient and care provider via a wrist strap (button, or clip-on broach, etc.) or other means, or perhaps implanted with bio-compatible RFID tags. The RFID tag can be affixed either directly or integrated to a smart medical device/apparatus 110, or as part of a wireless electronic computer module and associated electronics (or even RFID system on a chip). The point of care provider would have an RFID reader integrated or interfaced to a device (mobile handheld PDA or portable wired/wireless computer, or similar wearable device) capable of communicating with the smart medical compliance system and main patient data base and records system. In this scenario, the care provider would confirm the identification (ID) of one's self and the patient by a close proximity (near field communications, NFC) RFID scan, or otherwise, and in a similar manner, scan the smart medical device/apparatus prior to administering or performing the medical procedure. The mobile hand held computer, personal digital assistant (PDA), or portable mobile wired/wireless computer, would corroborate the correct correspondence among the care provider ID, the smart medical device/apparatus ID, and that of the ID of the patient.

In an Operator-Responsible Mode, in the event that the ID of the care provider, patient, and smart medical device/apparatus were correct, that is, confirmation made between the RFID of the care provider, patient, smart medical device (with corresponding medical content/prescription), and the overseeing smart medical compliance system, then a “go” alarm (audible, visual, text, or otherwise) condition exists. This information is then conveyed to the care provider, so that he/she can proceed with the act of administration. On the other hand, in the event of an ID mismatch, or incorrect compliance determined by the smart medical compliance system, a notification also in the form of an alarm would indicate and convey the mismatch, indicating a “no-go” condition. At this point, it is the care provider's responsibility to cease the administration attempt immediately to prevent mishap and re-assess the task at hand.

In a Fail-safe Mode, a second fail-safe mechanism can be included that would activate/deactivate a shut-off device, or latch, located on or within the smart medical device/apparatus itself, that would be enabled/disabled and/or moderate the administration according to the instructions received by the mobile hand held PDA computer or portable wired/wireless computer (near-real time information) and perhaps in conjunction with the smart medical compliance system. The activation (or lack of) would depend on the ID corroboration of the set—that being the smart medical device/apparatus, patient, and care provider, and the information communicated by the smart medical compliance system. For example, confirmation could be made by the portable wired/wireless computer of the care provider such that the RFID enabled device on a smart medical device/apparatus (smart syringe) would activate an unlock mechanism—permitting the commencement of injection of fluid to a patient. In the event that the smart syringe, patient, and care provider ID did not match, the smart syringe would not be enabled—and effectively remain shut-off or locked.

In either of these modes of procurement, the time-stamp and event would be logged by the mobile or portable wired/wireless computer and smart medical compliance system 108 (black box 101b), and subsequently, the event recorded and stored in its data base and/or the main patient data base and records system.

### Physical Realization of Medical Device/Apparatus Latch/Lock Mechanism:

The latch mechanism can include any of the following but are not limited by these:

1. A simple rotation of a mechanical latch that can be in a variety of states. For example, a red indicator indicating do not use, a yellow indicator indicating that the prescription is in a prepared state, and a green indicator indicating that the smart medical device/apparatus is now unlatched and ready to use.

2. An electromechanical latch, a stop or friction mechanism (Solenoid). This would be enabled by a separate power mechanism such as an on board battery, induced power through RFID device, piezo-electric effect, chemical, electrostatics, magnetics, or other mechanical to electrical transfer device.

3. An electrochemical latch activated by electromagnetic energy (eg., Artificial muscle, Magneto-Rheological, electrochemical).

4. A shape memory alloy latch activated by an electrical current or direct heat. This would be enabled by a separate power mechanism such as an on board battery, induced power through RFID device, piezo electric effect, chemical, electrostatics, magnetics, or other mechanical to electrical transfer device.

5. A fuse or anti-fuse activated by current or electromagnetic, or chemical (possibly pyrotechnics) energy.

6. A cantilever activated by electromagnetic energy (or heat, or light).

In any case, the main idea is to use the RFID device to either directly or indirectly drive a “switch” to activate a latch/lock, thus enabling or disabling the syringe.

### Secure Channel Encryption for Smart Medical Device/Apparatus Communication

The overall process of recording these (and previously described) operations is analogous to the data logging employed in the aviation industry utilizing a “Black Box.” Other types of failure modes can be handled in a similar fashion under the guidance and instruction from the information management and compliance system. Such transactions (device status, time stamps, authorization, operations, etc.) and data storage can be made cryptographically secure preventing alteration or modification.

The tracking and interoperable communication of smart medical devices/apparatus deployment could include suitable secure encryption methods. This will ensure reliable and confidential delivery and handling of critical data and secure control and actuation signals (or communication channels) for smart medical devices/apparatuses and operations management.

### Reading of Multiple RFID Tags

There may instances when several RFID tags may have to read at the same time, by the correct positioning of the handheld RFID Reader. The process of reading several RFID tags may be critical in order to corroborate the medical compliance and warrant the proper operation of the smart medical device/apparatus and its ability to execute (deliver or administer) its services (treatment, testing, or monitoring). The smart medical compliance system and method 100a (and its accompanying hardware, software and middleware) incorporate this feature seamlessly in its operation.

### Alternative Communication and Activation Means:

Smart medical devices/apparatuses can alternatively function by providing identification via an RFID tag, RFID System on a chip, Rubee, or HP-spot technology, etc. (all Radiofrequency communication technologies) while the actual indicator, locking/unlocking, and other control and information transmission signals, could be communicated to the smart medical device/apparatus using an auxiliary communication channel. Such channels could be standards such as 802.11x, 802.15.4, Bluetooth (Ericsson), Wibree (Nokia), ZigBee, HomeRF, Ultrawide band, 802.16, Wireless USB, or a proprietary ad hoc wireless communication means. Furthermore, it should be noted that wireless infrared could also be the means of communication for interoperability, control, and information gathering. In effect, a smart medical device/apparatus can be interrogated using an RFID Reader while the control and actuation can be affected using an alternative communication channel or protocol. The preferred incarnation, however, is to obtain identification, status, and control, or actuate the syringe itself, using the bi-directional RFID communication capability via an RFID reader.

In one instance, a micro RFID Reader/electronics could be collocated with the RFID tag and accompanying smart medical device/apparatus. Once the RFID tag is interrogated by the operator, and programmed accordingly, the response of the RFID tag could be read by the collocated micro RFID Reader/electronics and the appropriate action taken. The purpose of the collocated micro RFID Reader/electronics would be to support the requirement of flexibility in the interfacing of various sensors and actuators, thereby improving or extending the interfacing capability of the apparatus. This type of deployment could capitalize on standard off-the-shelf commercially available RFID tags whose standard electronic characteristics are either insufficient or unavailable to support or conform to the desired requirements.

### Smart Medical Devices/Apparatuses

There are many point of care medical devices that can be made “smart.” This would entail adding a certain amount of “intelligence” or capability for the purpose of increasing the level of functionality, scope, and application domain of the device. In doing so will make the device more suited to the task at hand. They include, but are no exclusive to, smart medical containers, smart clamps, smart valves, smart syringes and pipettes, smart couplers, and smart catheters. In the this, which can be transported by hand . . . .

### On-Board Visual and Audio State Indicators:

A visual icon/graphic based or color coded indicator could be incorporated into the smart medical device/apparatus 110 serving as a “state” indicator for maintenance, replacement, or authorized (or unauthorized) access to an insecure latch, lockable-latch, or activation of the smart medical device/apparatus control or actuator mechanisms. For instance, a Red color indicator could correspond to a locked state, while a Green indicator could correspond to an unlocked state, denying or allowing access, respectively. Flashing lights can be used to indicate ready (not-ready) status or actual occupied/busy/in the process of activation status. The color indicators themselves may be passive (color swatches) or active, using light emitting diode (LED), liquid crystal display (LCD), digital light processor (DLP), or other comparable display technology.

The smart medical device/apparatus 110 could also employ an audio indicator (i.e., speaker) incorporated into the smart medical device/apparatus 110 housing itself serving as an audio indicator for maintenance, replacement, or authorized (or unauthorized) access to an insecure latch, lockable-latch, or activation of the smart device/apparatus 110 control or actuator mechanisms. Reverberating audio can be used to indicate ready (not-ready) status or actual occupied/busy/in the process of activation status.

It should also be understood that a visual or auditory signal can convey information to care provider 114 that the correct patient 106 is selected, the correct “smart” medical device 110 is selected, or even if the correct tubing or line is selected, since they can be RFID or simply color tagged.

### Personal Digital Assistant (PDA) Mobile Portal.

It should also be noted that information can be conveyed to “authorized personnel” only through viewing screens and speakers of their personal computer, workstation, or mobile hand held or PDA devices 115, via signals transmitted through (hard wired, or wireless) communication channels. This can be accomplished so that the information is displayed in near-real time, whereby the operator has knowledge of the prevailing and pending processes and status, as the events occur, so that one may act or respond accordingly as one sees fit. In other words, this is the main human-computer interface (or portal) for conveying instruction and information about processes to the care provider 114. Furthermore, most of these devices have a key-pad, touch-sensitive screen, or stylist, for data enter and gaining access to information and database record systems.

Utilizing the display of a PDA 115 can indicate (instruct) the care provider 114, in near real-time, of any pending mistakes that can lead to adverse events, so that they can be avoided or amended altogether. Therefore, it is an important tool to be used by care providers for mitigating errors in medical administration by instilling corroboration and authentication protocol through the smart medical compliance system 100a. Color can be displayed on the PDA screen (as well as the text) of the item corresponding to a particular tagging of a medical device or supply. So, for instance, if a certain medical task calls for a smart syringe to connect to a certain piece of medical tubing/coupler, that is marked with a red tag on its end, then the word tubing and the color red can be displayed on the care provider's PDA display in order to guide the care provider in performing the correct operation. Moreover, the PDA's auditory alarm can also assist in this process/method as well.

### Universal Smart Key for Medical Applications:

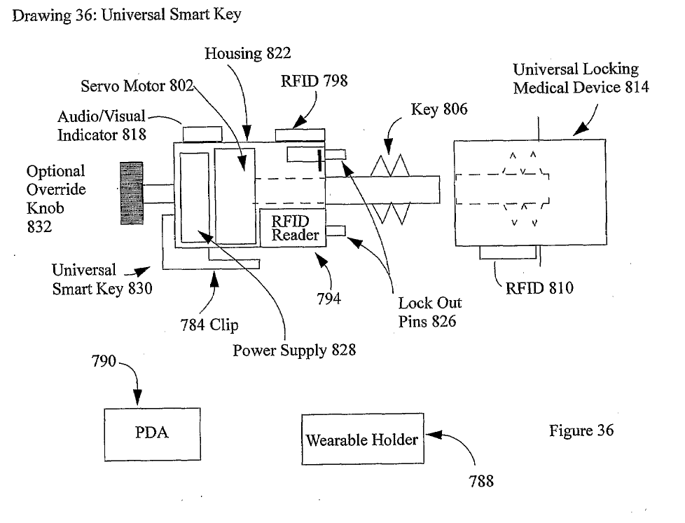

The universal smart key illustrated in FIG. 36 contains all the necessary features and functionality necessary to perform the operation of enabling/opening or closing of a lock that is universal and either affixed or incorporated into all of the said smart medical devices/apparatuses. This will make the manufacturing of smart medical devices much simpler since the only overhead is in the incorporation of a mechanical lock to be accessed by an electronic key. The smart medical devices themselves, however, will still contain and RFID tag 134 for identification and corroboration purposes. It contains an RFID tag 798, RFID Reader 794, renewable power source, drive servo/motor, protruding mechanical key (which is driven by the servo mechanism), visual and audio indicators, status sensors, and a compact housing (or dongle) to be attached/detached from a cradle—typically on or near the mobile PDA or hand held computer 790. The RFID enabled smart key fits into any and all smart medical devices/apparatuses, and can only operate (open, or close) the lock of the smart medical device if corroboration occurs. The smart key also incorporates an RFID Reader used to determine if the identification of the correspondingly “pre-mated” smart medical device is indeed the intended one by the operator. In this way, if the smart key is presented to the medical device, and corroboration/authentication occurs, the knob on the smart key will be free to rotate. Hence, this “permits” the lock to open (either manually by the operator, or automatically, depending on the embodiment), thereby allowing the operator to gaining access to the smart medical device's/apparatus's contents (as in a smart medical container), or in another manifestation, allowing the medical task (service, procedure, operation) at hand to proceed. It should be noted that the “programming” of the smart key is performed by a hand held PDA or mobile handheld device 790 with wireless communications and operated by the care provider.

FIG. 36 is a diagram illustrating a universal smart key and lock mechanism. FIG. 36 shows the PDA 790, a wearable holder 788, capable of affixing the universal smart key 830 with clip 784. The universal key includes a housing 822, RFID reader 794, RFID tag 798, key 806, lock out pins 826, power supply 828, audio/visual indicator 818, and servo motor 802. The lock out pins or similar prevent the key 806 from being inserted into the universal locking medical device 814. The universal locking medical device 814, is affixed with an RFID 810. The RFID reader 794 is capable of reading the RFID 810 of the universal smart lock 814 and corroborating this with its RFID 798 and interacting with the PDA 790 to confirm that the key is authorized to open the universal smart locking medical device 814. Upon authorization, the lock out pins 826 no longer prevent the key from accessing the lock. The universal smart key also has an optional override knob 832 allowing manual operation.

### Smart Container for Medical Applications

The following description is that of an intelligent or “Smart”-container (to be used in a method and system) designed for improving delivery of medication, medical supplies, or medical devices/apparatuses at the patient point of care (including surgery). At present a non negligible number of medical incidences (adverse events, sentinel events, and near misses) occur comprising of errors and accidental and/or incorrect drug administrations to patients as a result of ineffective identification and control. The invention described here therefore relates to medical reconciliation and compliance at the patient/care-provider delivery interface and/or pharmacological preparation and dispensing point. The container and its deployment address identification and/or control. Identification is accomplished with the aid of radio frequency identification (RFID) while the control is enabled through a mechanism that can be activated to prevent improper access, or facilitate error-free dispensing and/or administration of medication and/or medical supply or apparatus. The invention incorporates an RFID enabled electromechanical lockable latch enabling the opening and/or closing of an access gateway to the content of the container. The RFID tag on the container can be either active or passive, and the electromechanical latching/locking communication and control can be derived from the interaction of the Reader and the RFID tag or associated electronics (including RFID System on a chip technology). For example, an enable control signal received from the Reader could be used to unlock and/or open the container.

An instance of the execution of the medical reconciliation and compliance platform could entail the following (although not limited to following):

### Dispensing of Medical Content: Preparation and Packaging

At the pharmacological preparation (dispensing) point, and/or central medical supply unit, medical content could be identified and placed in an RFID enabled portable or mobile container. The RFID enabled container and content could be registered for subsequent tracking: this comprises identifying and initializing (locking) the container and/or content. At this stage, any mis-packaging can be detected and circumvented prior to discharge. The tracking and interoperable communication could include suitable encryption methods ensuring reliable and confidential delivery and handling of critical data.

### Transport

The container (along with medical content) could then be transported to the patient point of care (or surgery) via a means consistent with conventional medical content distribution. As such, a mobile cart (with secure access) is deployed to seamlessly accommodate such RFID enabled containers. By the strategically placing of RFID readers along the path of transport, time stamp and geographic tracking of entire cart content could be established.

Point of Care medical practice

It is envisaged that a point of care provider could have an RFID reader and a device (portable wired/wireless hand held computer) capable of communicating with the RFID reader and concurrently capable of communicating with the main patient data base and medical records of a health information system. This communication could be provided through a wireless or wired network, intranet, Internet, cellular or telephone system. With respect to the hand held computer, the RFID reader could be attached, built in, or detached, with wired or wireless communication or stand alone capability. Furthermore, the care provider could interrogate the RFID tag of the patient (or alternative identification) and corroborate this with the RFID tag of the container—containing the medical content itself. Upon corroboration (matching the correct identity of patient with the medical content) the container could be electromechanically unlocked (or unlatched) and the medical content retrieved. This process provides information including time-stamp that may optionally be logged back to the main patient data base and medical records system. It should be noted that the RFID reader can not only read the RFID tag of the lockable container but could also interrogate the medical content explicitly (whilst encapsulated) if the medical content where equipped with an RFID tag.

### Smart Medical Container: Recycling—Disposal, Sterilization, and Reuse:

The Smart Medical Container operational components may require sterilization for safe reusability. Proper handling and disposal protocol (i.e., Bio-hazard compliance) may require that a Disposal Reader log the RFID tag of the smart medical container and time-stamp the disposal, sending this information back to the main information management or records system. Even the reconstitution of the smart medical container for re-use may require the time-stamping in its preparation prior to redistribution. A secure tamper-proof inlay indicator may be affixed (if required) at this point of preparation.

As an added feature, the RFID enabled smart container can also be used to dispose of any residual, remaining, or expended medical content, such as medications (pill, powder, or liquid), medical waste, medical supplies, medical devices/apparatuses, or combinations thereof, or potential (bio-) hazardous material not necessarily previously RFID tagged. In this manner the smart container can be “programmed” or initialized to identify content for proper disposal or re-sterilization.

The RFID enabled lockable latch container can also be tailored to contain medications (pills, powder, or liquid), or other pharmacological preparations, and/or other medical supplies, substances, and/or apparatuses, thereby making it usable for a wide variety of content. The RFID enabled container (or components therein) could also be made sterilizable for reusability.

Moreover, the RFID enabled container can also be used for inventory or storage purposes or for the easy identification of any medical content or apparatus.

An extension could include a box with a RFID device capable of latching the container as well as a RFID Reader integrated on or in the container capable of interrogating the contents of the container itself. In this manner the RFID tag (and therefore the contents) can be recorded when the content is placed in the container and simply be interrogated without opening the container.

An analogy of the RFID enabled container is similar to any system where items are stored in larger containers. These are typical of transport and communication systems involving layered architectures. The RFID enabled container is capable of providing secure storage for medical content, whether the medical content itself is RFID tagged or not. The secure storage is provided by way of a lockable mechanism or indicator and a means of tracking the container itself with its RFID tag.

The actual RFID enabled latchable container can manifest itself in a variety of forms, such as, although not limited to that of the following: a bottle; a two piece separable cavity; or a cylindrical or rectangular parallelepiped container. The opening of the container can be at the end, or on a side, or in the middle, as in a split two-piece cavity design. Other embodiments may include: a 2 piece (split) container design which separates (or breaks in two) at a latching/locking point; a “bag” with a lockable zipper; and a tear away package (with an RFID security inlay). The container can include a clip such that the container can be attached to an auxiliary apparatus or belt. The container can also be stackable such that many can be easily transported and/or read by a generalized RFID reader within an operable time window whilst within the same proximity (as in transport, or for inventory purposes).

The function of the RFID enabled container device would serve to unlock and/or open the container: in one instance, the RFID enabled device could supply an indication that the container could be manually opened; while in another instance, the RFID would serve to unlock the container as well as to open the container itself, thus exposing its contents.

An instance of a more generalized RFID enabled container could include the one or more of the following features, although not limited to these: an RFID tag or device for identification of the container, a microcontroller/computer with or without auxiliary radio frequency RF communication, an electromechanical or mechanical latch, a door or shutter that can have a mechanically assisted opening device, a disposable or rechargeable detached or attached power source to facilitate operation and communication, an RFID reader for content interrogation, a Peltier or similar heating or cooling device for environmental control, a disposable or sterilizable reusable inner liner for securing contents and/or the container itself sterilizable.

This portable container could be used for a myriad of applications where unlatching the container could be enabled by a limited number of persons on an access control list. These applications could include firearms, keys, cash, or valuables, etc. The role in the medication reconciliation is obvious with the additional benefit that it can be more easily integrated into a legacy system.

The Smart container could also be equipped with a Biometric user identification device ensuring only authorized access to its medical contents.

A color coded indicator is incorporated into the Smart container serving as an indicator for authorized (or unauthorized) access to its medical contents. In a manual mechanical operating mode, the latch can be opened for egress, or maintained in a locked state. Conversely, in an automated operating mode, the latch is electromechanically, or otherwise, by mechanical means, opened automatically for egress, or maintained in a locked state. For instance, a Red color indicator corresponds to a locked latch state, while a Green indicator corresponds to an open latch state, denying or allowing access, respectively. Furthermore, an intermediate color, such as Yellow, can be used to indicate that medical contents has been prepared and sealed in the container—ready for administration.

A security “trip-wire” (conductive strip/trace, decal, inlay, or pin, security seal) can be incorporated into the smart container affixed or attached by adhesive or glue (or other mechanical means) as a tamper-proof indicator. A passive or electrically conductive inlay strip or decal is placed over the latch/lock or attached to a movable/pivoting structure of a smart medical container which provides access (ingress, egress) to the gate/door or latch and the associated contents of the container. The state of the seal (secured or breached) can be read with an RFID Reader.

### Smart Clamp for Medical Applications

The following description envelops that of a clamp modified to include a Radio Frequency Identification (RFID) tagging device and interface with bi-directional communication. The design variations encompass several pinch-off embodiments but are not exclusive to the following forms of screw, cam, scissor, rotational (twist), push-type, lever-type, in-line latch, hinge, linear-actuator ram, or roller-actuator clamp. The capability therein incorporates an electromechanical controller that authorizes the operation of a manual or automatic mechanical clamp with lockable-latch and pinch-off mechanism, thereby modulating the flow of a powder, liquid, vapor, or gas through a collapsible tube or conduit as such. As described, the “smart clamp” invention incorporates an RFID tag and accompanying interface (in situ and/or external) in a method and system to control the mechanical operation of latching and pinching: i) manually enabling or precluding an instance of user operation of the pinch mechanism; or ii), automatically enabling or precluding an instance of electromechanically energized operation of the pinch mechanism. Hence, in addition to facilitating the flow by activating the pinch mechanism, this smart clamp assembly, method and system, incorporates a lockable latch which will prevent unauthorized, erroneous, or inadvertent operation of the clamp.

The RFID tag on the smart clamp can be either passive or active with an internal and/or external bi-directional electronic communication interface to control electromechanical circuitry. The RFID smart clamp can be identified with an RFID reader (in the manifestation of a mobile or stationary communicating electronic computing device) and used to “interrogate” clamp status. The communication and electromechanical control can be derived from the interaction of the RFID tag (and associated electronics) and the RFID reader—and perhaps an overseeing information management system. Upon identification, corroboration, and authentication, an “enable” or “disable” control signal received or transmitted from the interrogating RFID reader could be used to activate or preclude the latch and/or pinch mechanism associated with the RFID smart clamp, respectively. The RFID smart clamp can also include auxiliary sensor information that can be communicated to the RFID reader or vise versa and if necessary up the information management hierarchy for re-programming, time stamping, data collection, and/or evaluation. This sensor based acquisition can include information such as chemistry, temperature, humidity, pressure, flow rate, etc.

In effect, the RFID smart clamp assembly, method and system, is distinct in that it is used for both identification and as a means of control for the enabling or preclusion of flow of a powder, liquid, vapor, or gas through a deformable line or channel (conduit, tube, or hose). The manual user operated pinch mechanism inherently offers a high degree of application flexibility and simplicity in design without necessarily compromising functionality. This approach can therefore be particularly attractive in many instances since it may well capitalize on a concomitant reduction in overhead, ease of fabrication, and increased mechanical reliability. On the other hand, offering an automatic RFID triggered pinch regulating mechanism offers the potential for ease of field deployment and robustness. This implementation incorporates, but is not exclusive to those mentioned herein, an electromechanical solenoid, servo or motor, pneumatic/hydraulic (possibly Magneto-rheological) cylinder or motor, or temperature activated shape memory alloy drive—acting upon a screw, cam, ram, pincers, or the like. In recognition of these renditions of both manual and automatic pinch regulating constricting mechanisms, they yield a myriad of useful clamp variants to be used within the context of the RFID smart clamp.

One instance of deployment of an RFID smart clamp (used in a medical setting) could be to safely gate the release of a fluid as would be the case for Intravenous (IV) or Infusion Therapy. In another instance of deployment, the RFID smart clamp is applicable to the handling or management of industrial chemicals. In general, these include volatile and/or hazardous powders, liquids, vapors, and gases. Where regulation and safety standards necessitate proper handling and mixing protocols, as such, the RFID smart clamp could be used in various medical, industrial, commercial, or residential applications.

There are several in-field embodiments of design pertaining to the definitive actuation or modulation (or lack thereof of an RFID smart clamp pinch mechanism: in one mechanical instance of operation, the RFID smart clamp would simply provide a visual or audio status indicator approving/disapproving the manual operation (open or close) of the clamp pinch mechanism; in another mechanical instance of operation, the RFID smart clamp could physically unlock a latch mechanism that would permit the manual operation (open or close) of the clamp mechanism; finally, in an electromechanical instance of operation, the RFID smart clamp could physically unlock a latch mechanism and provide the actual electromotive means that would automatically modulate (open or close, potentially with cam or ram specified variations therein) the clamp pinch mechanism.

The actual RFID smart clamp physical deployment can manifest itself in a variety of forms, although not limited to the following: a clamp that is in effect “clamped” or fastened over a deformable line or channel (conduit, tube, or hose); or, a clamp whereby the deformable line or channel (conduit, tube, or hose) is inserted through the apparatus itself. As such, these basic variations include clam-shell and in-line versions, respectively.

The primary function of the RFID smart clamp would serve to restrict the flow of a powder, liquid, vapor, or gas through a deformable channel. However, in the field, under temporal command and control, an RFID smart clamp would provide for the means of identification of the clamp itself, an indication of the actual state of restriction—or variations thereof, and the intended position or state of the pinch mechanism (opened or closed) to be either manually or automatically accommodated. With an automatic capability, the RFID smart clamp would “automatically” serve to pinch the tube itself by way of an electromotive mechanism, thus allowing or restricting the flow of a powder, liquid, vapor, or gas through a deformable channel.

In general the RFID smart clamp could include one or more of the following features, although not limited to these: an RFID tag or device for identification of the smart clamp; a microcontroller/computer with or without auxiliary radio frequency (RF) communication; an electromechanical or mechanical flow constricting (limiting) mechanism; and/or, a disposable or rechargeable detached or attached power source to facilitate operation and communication in the event the power were not provided by the RFID interrogator. Instances of these components and others are illustrated in several smart clamp variants in the attached drawings.

The RFID smart clamp could be used for a myriad of applications where restricting of a flow would be useful. The smart clamp could also be enabled by a limited number of persons on an access control list preventing inadvertent or malicious operation of the clamp by unauthorized personnel. This access control list would be moderated, controlled, and maintained by an overseeing information management system.

In the event of device failure or tampering, the smart clamp will recognize the state or instance of anomaly using onboard sensors and logic and initiate a failsafe protocol to circumvent dangerous administration or utilization.

It is envisaged that the clamp operator at the point of actuation would have an RFID reader with a personal digital assistant (PDA) or other portable or mobile wired/wireless hand held computer (integrated or stand alone) capable of communicating with the smart clamp and concurrently capable of communicating with the main plant information management system. This later communication could be provided through a wireless or wired network, intranet, Internet, cellular or telephone system. As previously mentioned, with respect to the hand held computer, the RFID reader could be attached, built in, or detachable, with wired or wireless communication or stand alone capability. The protocol of operation calls for the clamp operator to interrogate the RFID tag or smart clamp and upon corroboration (matching the correct identity of the clamp with the desired actuation state) the smart clamp could be electromechanically unlocked (or unlatched) and the pinch mechanism enabled or prepared for actuation. If the smart clamp is manual in nature (mechanical instance) it is left to the operator to perform the actual pinch-off function using the built in or keyed manual actuator. On the other hand, if the smart clamp is automatic in nature (electromechanical instance) the actual pinch-off function is performed automatically without operator intervention or assistance. The process described above can also provide services including time-stamps, data collection, and sensor information that may optionally be logged back to the main plant records system.

A failsafe protocol can also be affected within the scope of the present method and system. In the event of smart clamp device failure, the operator has prior knowledge and procedures for overriding the malfunctioning smart clamp without compromising intended purpose, operation, and functionality. The smart clamp performs self test diagnostics periodically, upon power up, or query. In this manner, any anomaly will be reported to the operator for override, repair, or intervention. The onboard intelligence of the smart clamp itself will provide an audio or visual warning, or through wireless communication, provide a warning to the hand held computer notifying the operator of a hardware failure. The operator may now invoke an override procedure to manually bypass the failure and continue with intended clamp function in compliance with failsafe protocol. The overall operation is not compromised since the failure mode has been recognized and recorded, thus allowing steps to be undertaken to repair or replace the defective smart clamp. This process is overseen by the information management and compliance system to conform to standards for minimizing the presence of a defective smart clamp in the field. The compliance conforms to requirements within each field of application and deployment. The purpose is to safeguard against erroneous, unauthorized, malicious, or accidental use, and moderate the handling or management of defective or obsolete devices. The actual override procedure is performed by an authorized operator utilizing a manual actuator or override key. This operation is also monitored and recorded by the smart clamp and communicated via the handheld computer to the information management and compliance system.

Other types of failure modes would be handled in a similar fashion under the guidance and instruction from the information management and compliance system.

The Smart Clamp operational components may require sterilization for safe reusability. Proper handling and disposal protocol (i.e., Bio-hazard compliance) may require that a Disposal Reader log the RFID tag of the smart clamp and time-stamp the disposal, sending this information back to the main information management or records system. Even the reconstitution of the smart clamp for re-use may require the time-stamping in its preparation prior to redistribution. A secure tamper-proof inlay indicator may be affixed (if required) at this point of preparation.

The attached figures illustrate various instances of the smart clamp and modes of operation. The drawings encompass several pinch-off embodiments: screw-type (drawings 2 and 3); cam-type (drawings 4 and 5); scissor-type (drawing 6); rotational-type (drawing 7 and 8); push-type (drawing 9); lever-type (drawing 10); in-line latch-type (drawing 11); hinge-type (drawing 12); linear-actuator ram-type (drawings 13 and 14); and, roller-actuator-type (drawings 15 and 16).

These smart clamps all contain lockable-latch mechanisms (but could simply contain an insecure latch) with pinch-off mechanisms that are either manual (drawings 2, 4, 6, 7, 9, 10, 11, 12, 13, and 15) or automatic (drawings 3, 5, 8, 14, and 16) in function. As illustrated, the instances of manual smart clamps have manually operated actuators which serve to activate the pinch-off mechanism. Contrastively, the automatic smart clamps have an automatic (electromechanical) pinch-off mechanism to serve the same functionality.

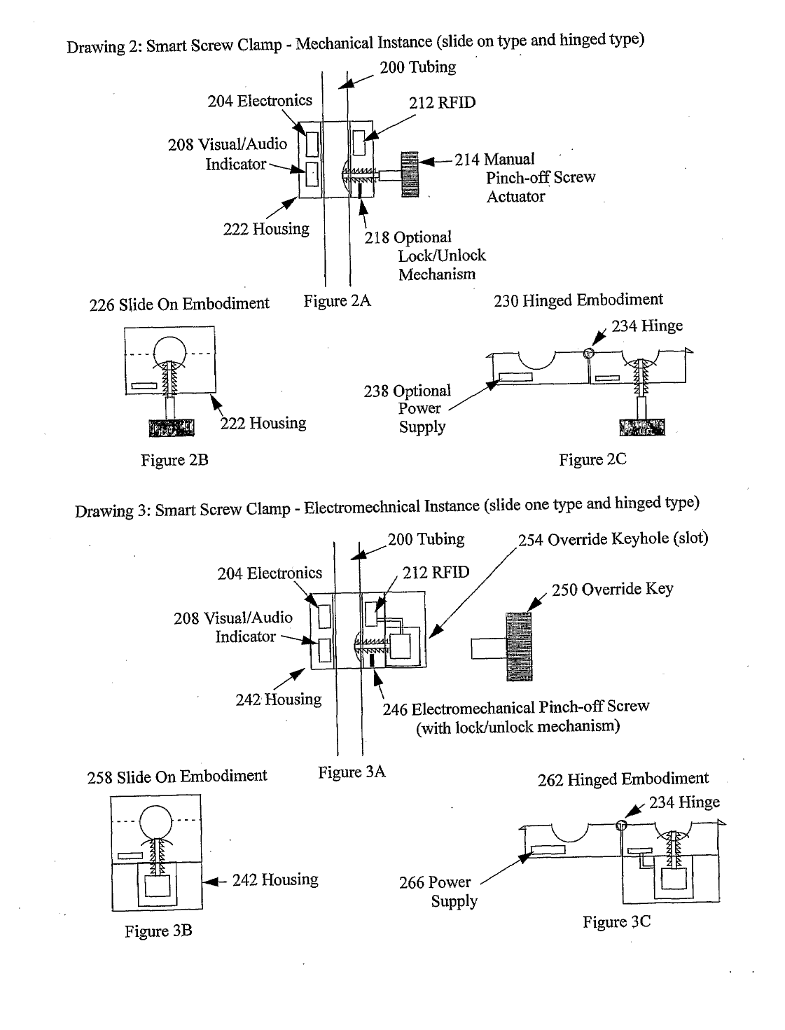

In the series of Figures numbered 2 is a diagram illustrating Smart Screw Clamp—Mechanical Instance (slide on type 226 and hinged type 230). FIG. 2A shows the tubing 200 encased by the RFID screw clamp consisting of the RFID 222, manual pinch-off screw actuator 214, an optional lock/unlock mechanism 218, encased in a housing 222, including a visual/audio indicator 208 and associated electronics 204. In the hinged embodiment the hinge 234 allows the clamp to be clamped onto the tube 200 while in slide on embodiment 226 the tube 200 is slid into the clamp housing 222.

In the series of Figures numbered 3 is a diagram illustrating Smart Screw Clamp—Electromechanical Instance (slide on type 258 and hinged type 262). FIG. 3A shows the tubing 200 encased by the RFID screw clamp consisting of the RFID 212, electromechanical pinch-off screw 246 (with lock/unlock mechanism), encased in a housing 242, including a visual/audio indicator 208 and associated electronics 204. In the hinged embodiment 262 the hinge 234 allows the clamp to be clamped onto the tube 200 while in slide on embodiment 258 the tube 200 is slid into the clamp housing 222. 266 is the power supply.

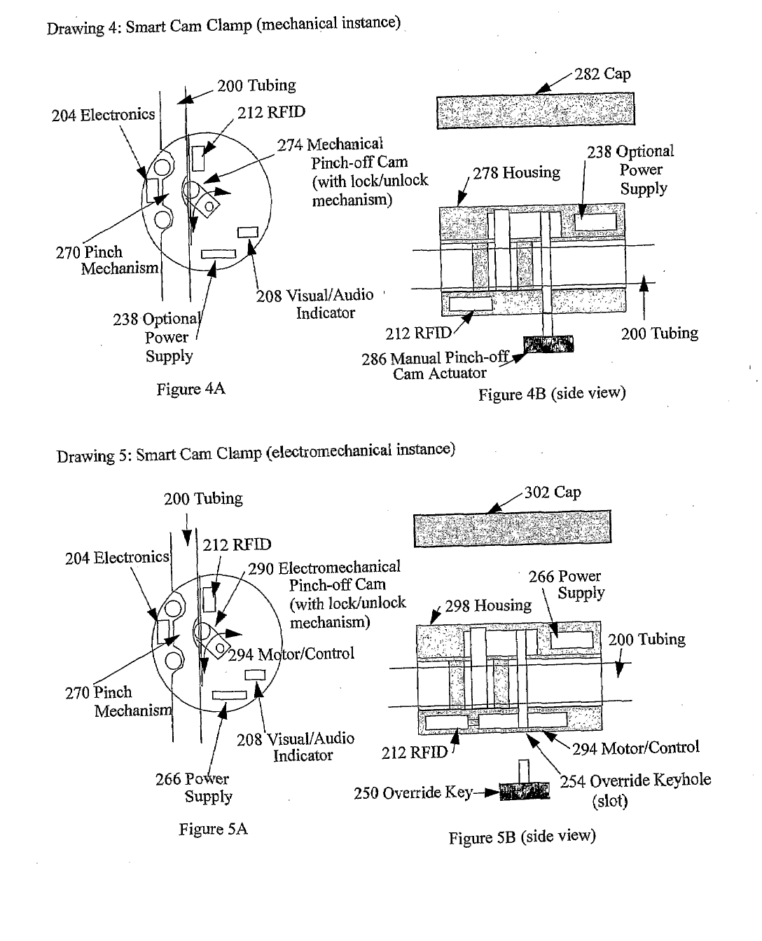

In the series of Figures numbered 4 is a diagram illustrating smart cam clamp (mechanical instance). FIG. 4A shows the tubing 200 encased by the RFID cam clamp consisting of the RFID 212, mechanical pinch-off cam 274 (with lock/unlock mechanism), encased in a housing, including a visual/audio indicator 208, optional power supply 238, and associated electronics 204. FIG. 4B shows a side view of the embodiment with housing 278 and cap 282. The manual pinch-off cam actuator 286 allows the cam to be impressed onto the tube 200 thereby blocking flow.

In the series of Figures numbered 5 is a diagram illustrating smart cam clamp (electromechanical instance). FIG. 5A shows the tubing 200 encased by the RFID cam clamp consisting of the RFID 212, electromechanical pinch-off cam motor control 294 (with lock/unlock mechanism), encased in a housing, including a visual/audio indicator 208, power supply 266, and associated electronics 204. FIG. 5B shows a side view of the embodiment with housing 298 and cap 302. The motor driven pinch-off cam actuator 294 allows the cam to be impressed onto the tube 200 thereby blocking flow. In the electromechanical instance an override key 250 is provided interacting with an override keyhole slot 254.

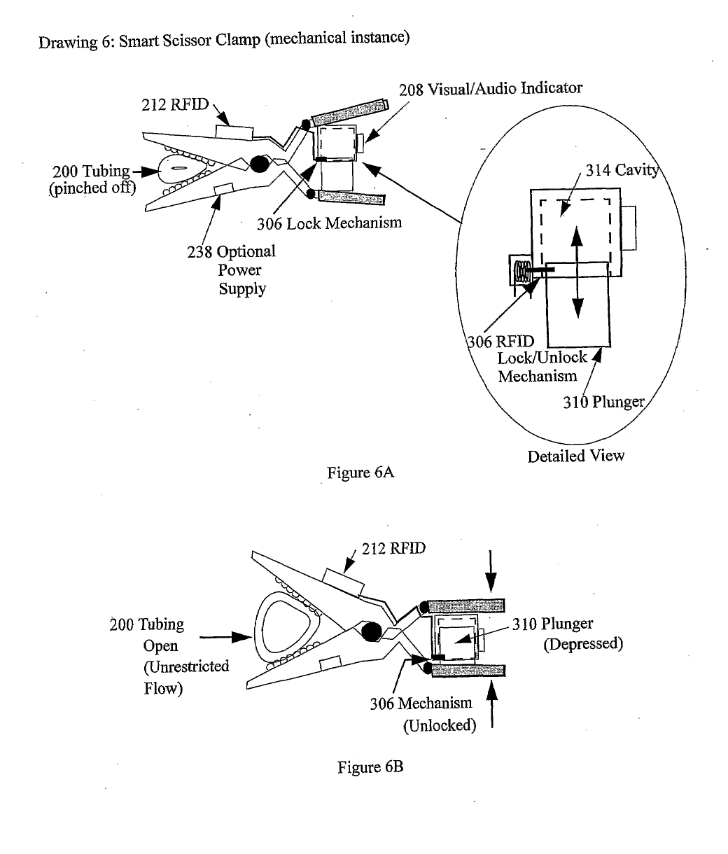

In the series of Figures numbered 6 is a diagram illustrating a smart scissor clamp (mechanical instance). FIG. 6A shows the tubing 200 pinched off by the RFID scissor clamp consisting of the RFID 212, mechanical lock mechanism 306 (with lock/unlock mechanism), including a visual/audio indicator 208, optional power supply 238. FIG. 6B shows a side view of the embodiment with lock mechanism 306 unlocked. In this case the plunger 310 is in a depressed state and the tubing 200 in an unrestricted flow state. 306 illustrates the RFID lock mechanism in more detail.

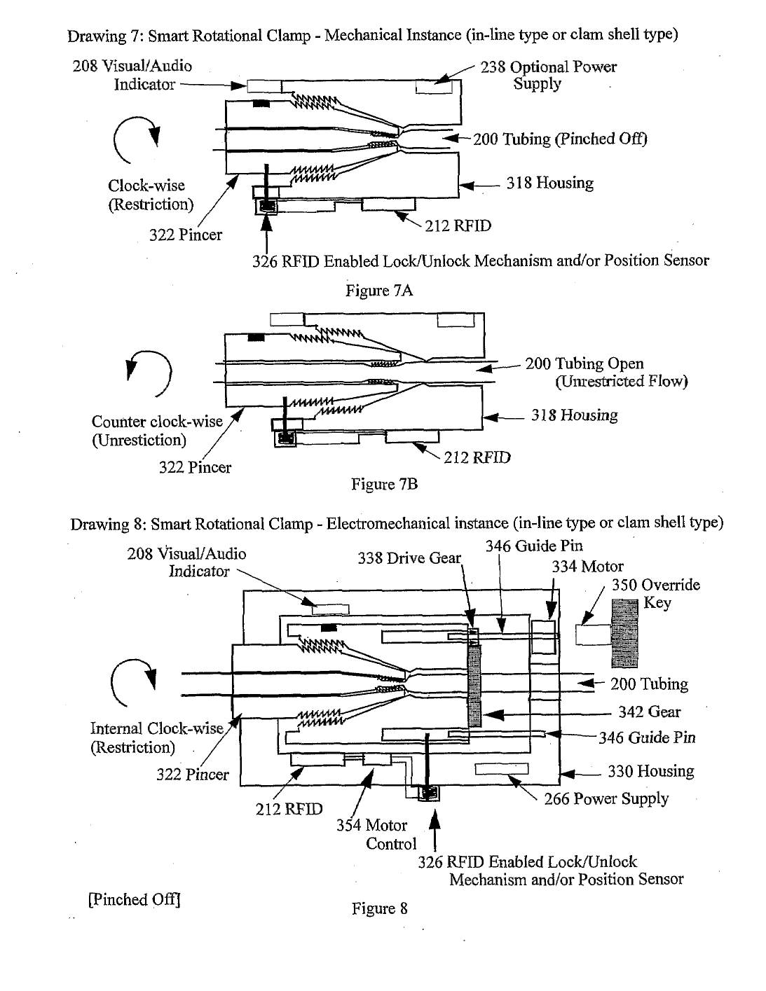

In the series of Figures numbered 7 is a diagram illustrating a smart rotational clamp—mechanical instance. FIG. 7A shows the tubing 200 pinched off by the RFID rotational clamp consisting of the RFID 212, mechanical lock mechanism 326 (with lock/unlock mechanism and/or position sensor), including a visual/audio indicator 208, optional power supply 238 and housing 318. FIG. 7B shows a view of the embodiment with the rotational pincer 322 turned out. In this case the tubing 200 is in an unrestricted flow state.

In the series of Figures numbered 8 is a diagram illustrating a smart rotational clamp—electromechanical instance. FIG. 8 shows the tubing 200 pinched off by the RFID rotational clamp consisting of the RFID 212, RFID enabled lock mechanism 326 (with lock/unlock mechanism and/or position sensor), including a visual/audio indicator 208, power supply 266 and housing 330. A motor 334 controlled by motor controller 354 is activated driving a gear 342 forcing the pincer 322 to restrict the flow in the tube 200. Guide pins 346 keep the electromechanical clamp aligned.

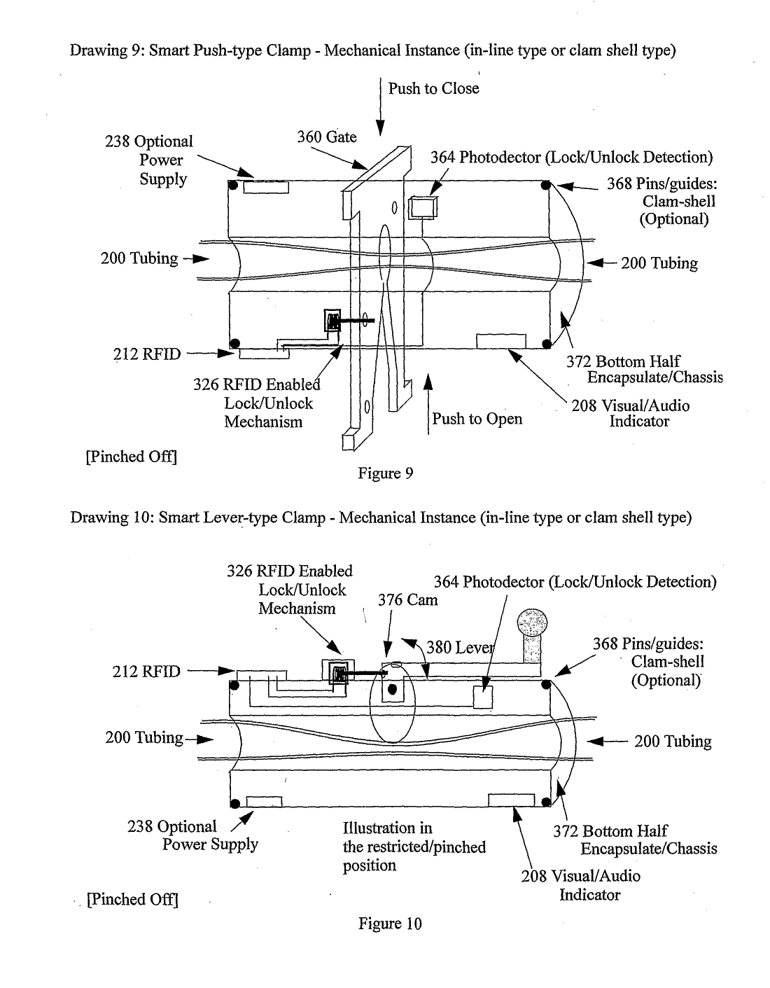

In the series of Figures numbered 9 is a diagram illustrating a smart push-type clamp—mechanical instance. FIG. 9 shows the tubing 200 pinched off by the RFID controlled pinch mechanism or gate 360 consisting of the RFID 212, RFID enabled lock/unlock mechanism 326, including a visual/audio indicator 208, optional power supply 238 and housing/chassis 372. Guide pins 369 keep the electromechanical clamp aligned when the embodiment is the clam shell type. The gate 360 is pushed to close to restrict the flow in the tube 200 when the RFID lock mechanism 326 is unlocked.

In the series of Figures numbered 10 is a diagram illustrating a smart lever-type clamp—mechanical instance. FIG. 10 shows the tubing 200 pinched off by the RFID controlled lever mechanism 380. The clamp consisting of the RFID 212, RFID enabled lock/unlock mechanism 326, including a visual/audio indicator 208, optional power supply 238 and housing/chassis 372. Guide pins 369 keep the electromechanical clamp aligned when the embodiment is the clam shell type. The cam 376 is levered to close or restrict the flow in the tube 200 when the RFID lock mechanism 326 is unlocked.

In the series of Figures numbered 11 is a diagram illustrating a smart in-line latch clamp—mechanical instance. FIG. 11 shows the tubing 200 pinched off by the RFID controlled latch mechanism 384. The clamp consisting of the RFID 212, RFID enabled lock/unlock mechanism 326, including a visual/audio indicator 208, and optional power supply 238. The latch clamp 384 is pinched to close or restrict the flow in the tube 200 when the RFID lock mechanism 326 is unlocked.

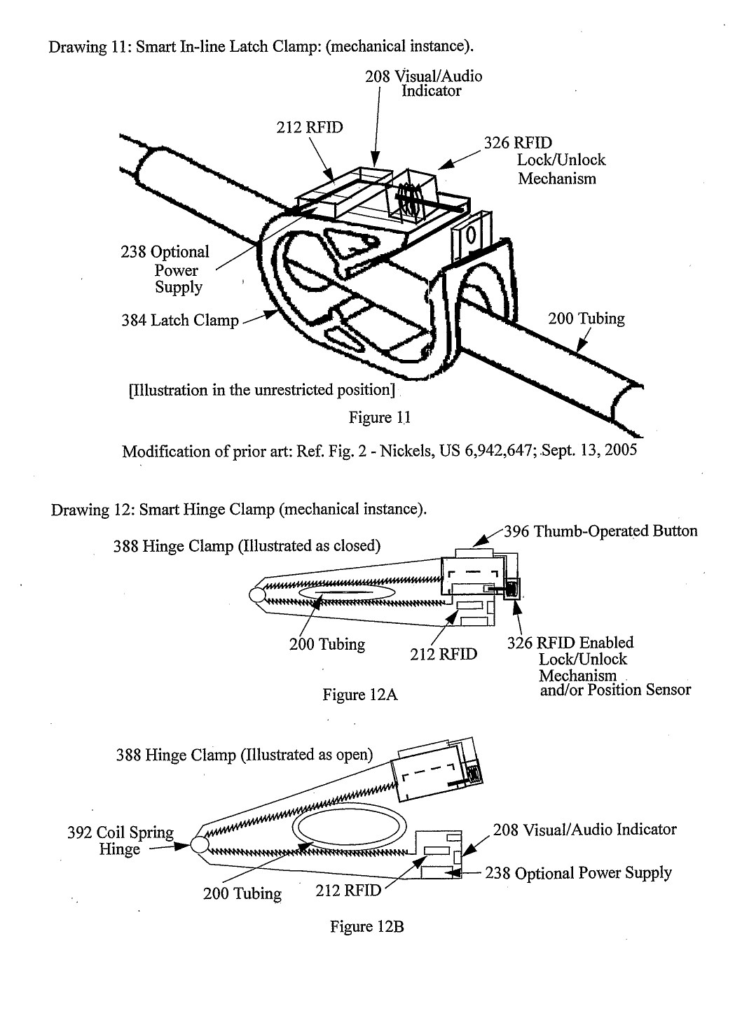

In the series of Figures numbered 12 is a diagram illustrating a smart hinge clamp—mechanical instance. FIG. 12A shows the tubing 200 pinched off by the RFID controlled hinge clamp mechanism 388. The clamp consisting of the RFID 212, RFID enabled lock/unlock mechanism 326, including a visual/audio indicator 208, and optional power supply 238. A thumb operated button 396 is used to unlock the hinge clamp 384 thus unrestricting the flow in the tube 200. The latch clamp 384 is pinched to close or restrict the flow in the tube 200 when the RFID lock mechanism 326 is locked.

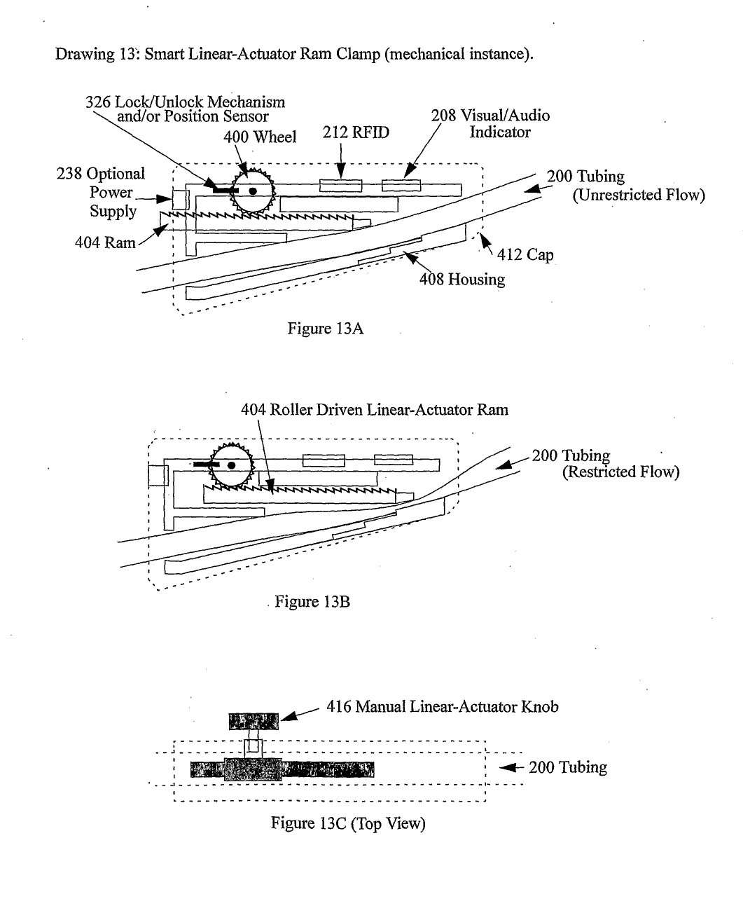

In the series of Figures numbered 13 is a diagram illustrating a smart linear-actuator ram clamp (mechanical instance). FIG. 13A shows the tubing 200 unrestricted by the thumb wheel 400 driven ram 404 controlled by the lock/unlock mechanism 326. The clamp consisting of the RFID 212, RFID enabled lock/unlock mechanism 326, including a visual/audio indicator 208, and optional power supply 238. A thumb operated wheel 400 is used to adjust the position of the ram 404 thus unrestricting or restricting the flow in the tube 200 when the RFID lock mechanism 326 is unlocked. FIG. 13B shows the linear actuator in the state of restricted flow. A housing 408 and cap 412 are also shown. FIG. 13C shows a top view illustrating the manual linear actuator knob or thumbwheel 416.

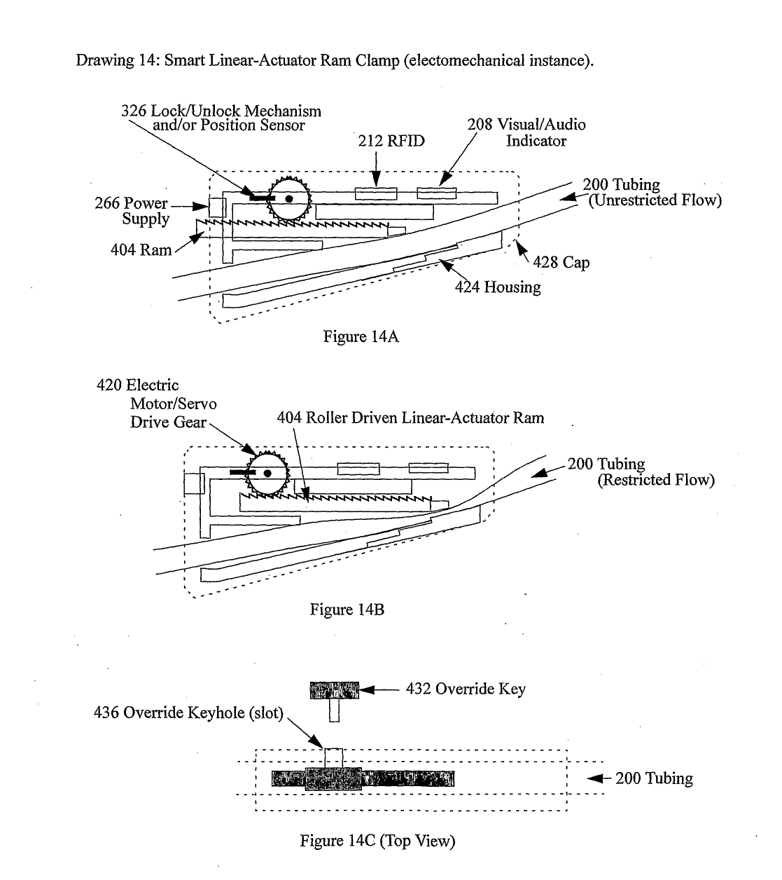

In the series of Figures numbered 14 is a diagram illustrating a smart linear-actuator ram clamp (electromechanical instance). FIG. 14A shows the tubing 200 unrestricted by the ram 404 controlled by the lock/unlock mechanism 326. The clamp consisting of the RFID 212, RFID enabled lock/unlock mechanism 326, including a visual/audio indicator 208, and power supply 266. An electric motor/servo operated wheel 420 is used to adjust the position of the ram 404 thus unrestricting or restricting the flow in the tube 200 when the RFID lock mechanism 326 is unlocked. FIG. 14B shows the linear actuator in the state of restricted flow. A housing 424 and cap 428 are also shown. FIG. 13C shows a top view illustrating a manual override key 432 and override slot 436.

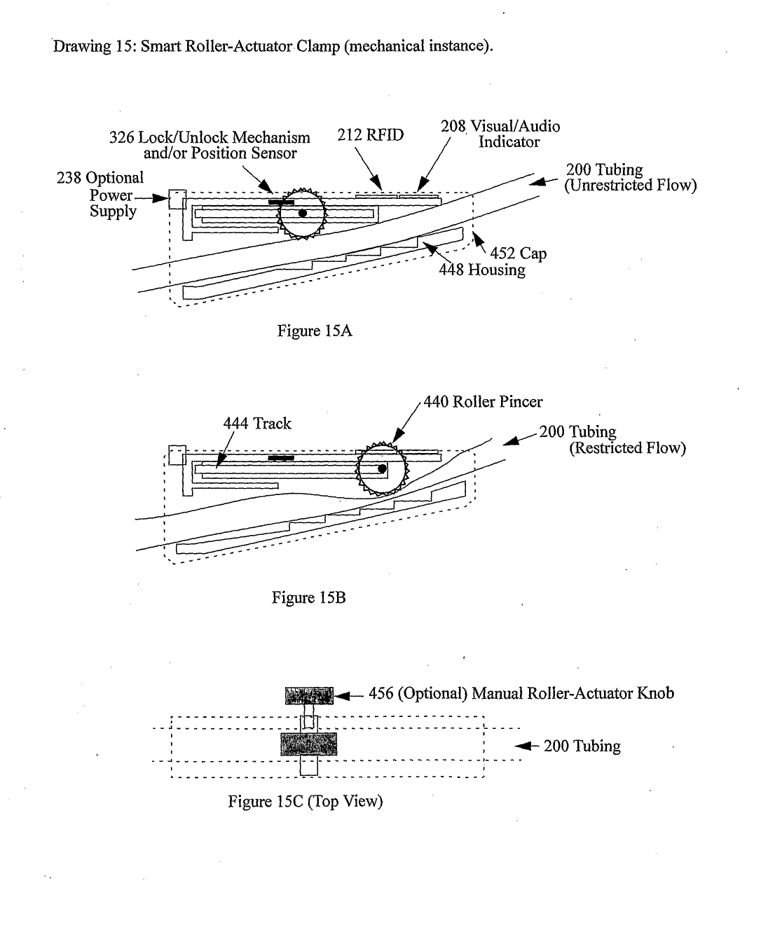

In the series of Figures numbered 15 is a diagram illustrating a smart roller actuator clamp (mechanical instance). FIG. 15A shows the tubing 200 unrestricted by the thumb wheel 426 controlled by the lock/unlock mechanism 326. The clamp consisting of the RFID 212, RFID enabled lock/unlock mechanism 326, including a visual/audio indicator 208, and optional power supply 236. A thumb operated wheel 400 is used to open or restrict the flow in the tube 200 when the RFID lock mechanism 326 is unlocked. FIG. 15B shows the roller actuator in the state of restricted flow. A housing 448 and cap 452 are also shown. The wheel is constrained to move in a track 444. FIG. 15C shows a top view illustrating the manual roller actuator knob or thumbwheel 326.

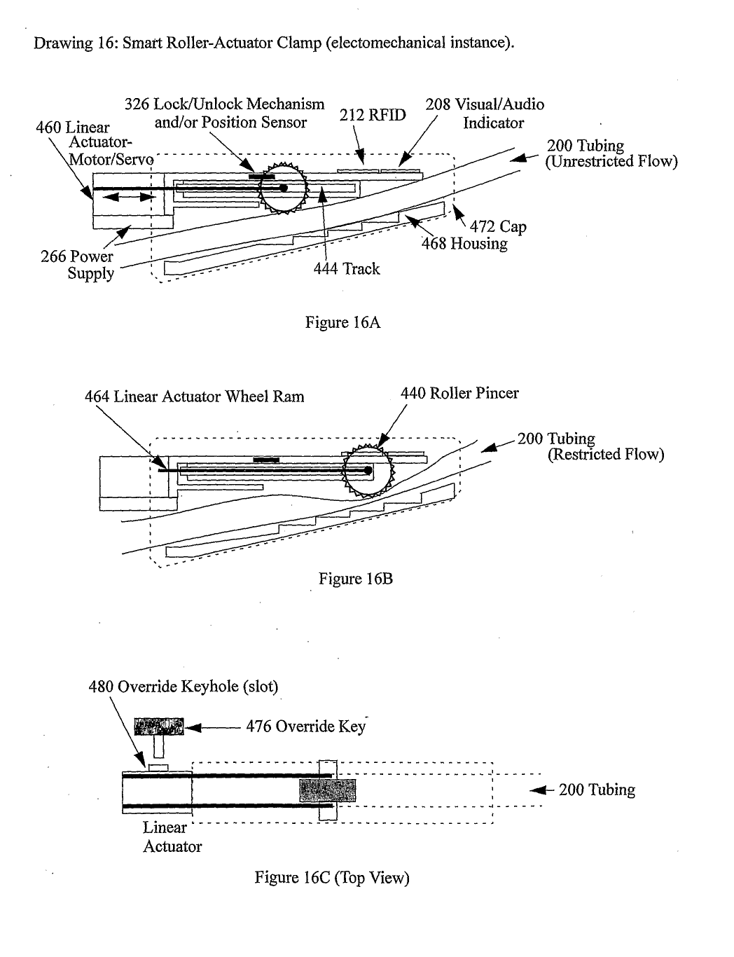

In the series of Figures numbered 16 is a diagram illustrating a smart roller actuator clamp (electromechanical instance). FIG. 16A shows the tubing 200 unrestricted by the roller controlled by the lock/unlock mechanism 326. The clamp consisting of the RFID 212, RFID enabled lock/unlock mechanism 326, including a visual/audio indicator 208, and power supply 266. An electric motor/servo adjusts a wheel thus unrestricting or restricting the flow in the tube 200 when the RFID lock mechanism 326 is unlocked. FIG. 16B shows the roller actuator in the state of restricted flow. A housing 468 and cap 472 are also shown. FIG. 13C shows a top view illustrating a manual override key 432 and override slot 436.

### Smart Valve for Medical Applications

A “conventional” valve is a manual or automatic (power assisted) mechanical device used to control the direction, volume, rate, and/or pressure of flow of a liquid, vapor, gas, slurry, or dry material (powder), through a passageway such as a line, channel, conduit, pipeline, hose, or chute.

The following “Smart Valve” description envelops this basic valve principle however offers much greater capability and purpose by including a Radio Frequency Identification (RFID) tagging device and interface with bi-directional communication. The smart valve design variations encompass several flow constriction (regulation) and housing embodiments. The capability therein incorporates a controller that authorizes the operation of a manual or automatic mechanical valve with lockable-latch and pinch-off regulating mechanism, thereby modulating the flow of a, liquid, vapor, gas, slurry, or dry material (powder), through a channel, conduit, pipeline, hose, or chute, as such. As described, the smart valve invention incorporates an RFID tag and accompanying interface (in situ and/or external) in a method and system to control the mechanical operation of a valve: i) manually enabling or precluding an instance of user operation of the valve regulating mechanism; or ii), automatically enabling or precluding an instance of power assisted (e.g., electromechanically) or energized operation of the valve regulating mechanism. Hence, in addition to facilitating the flow by activating the valve regulating mechanism (opening, closing, or modulating), this smart valve assembly, method and system, incorporates a lockable-latch which will prevent unauthorized, erroneous, or inadvertent operation of the valve. In the field, information will be available as to the status of operation (metrics, performance), maintenance, and serviceability.

The RFID tag on the smart valve can be either passive or active with an internal and/or external bi-directional electronic communication and microcomputer interface to control electromechanical circuitry. The RFID smart valve can be identified with an RFID reader (in the manifestation of a mobile or stationary communicating electronic computing device) and used to “interrogate” valve status. The communication and electromechanical control can be derived from the interaction of the RFID tag (and associated electronics) and the RFID reader—and, in some instances, an overseeing information management system. Upon identification, corroboration, and authentication, an “enable” or “disable” control signal received or transmitted from the interrogating RFID reader could be used to activate or preclude the latch and/or pinch-off regulating mechanism associated with the smart valve, respectively. The smart valve can also include auxiliary sensor information and memory that can be communicated to the RFID reader or vise versa, and if necessary, up the information management hierarchy for re-programming, time stamping, data collection, and/or (re)-evaluation. This sensor based acquisition can include information such as temperature, pressure, flow rate, viscosity, humidity, chemistry, Ph, etc.

In effect, the smart valve assembly, method and system, is distinct in that it is used for both identification and as a means of control for the enabling or preclusion of flow of a liquid, vapor, gas, slurry, or dry material (powder) through a passageway. The manual user operated valve pinch-off regulation mechanism inherently offers a high degree of application flexibility and simplicity in design without necessarily compromising functionality. This approach can therefore be particularly attractive in many instances since it may well capitalize on a concomitant reduction in overhead, ease of fabrication, and increased mechanical reliability. On the other hand, offering an automatic RFID triggered valve pinch-off regulating mechanism offers the potential for ease of field deployment and robustness. This implementation incorporates (but is not exclusive to those mentioned herein) an electromechanical solenoid, servo or motor, pneumatic/hydraulic (possibly Magneto-rheological) cylinder or motor, or temperature activated shape memory alloy drive—acting upon a shaft (screw) fixed to a plunger in the form of a gate, flap, rod, cylinder, ball, or acting upon a diaphragm, collapsible tubing, or the like, in a sealed housing or chamber. In recognition of these renditions (of both manual and automatic valve regulating constricting mechanisms), they yield a myriad of useful valve variants to be used within the context of the RFID enabled smart valve.

One instance of deployment of a smart valve (used in a medical setting) could be to safely gate the release of a fluid as would be the case for Intravenous (IV) or Infusion Therapy. In another instance of deployment, the smart valve can be used in handling, preparation, or management, of pharmaceuticals or industrial chemicals. In general, these substances include precious, volatile, and/or hazardous liquids, vapors, gases, slurries, and dry materials (powders). Where regulation and safety standards necessitate proper handling and mixing protocols, as such, the smart valve could be used in various medical, industrial, commercial, and residential/domestic settings.

There are several in-field embodiments of design pertaining to the definitive actuation or modulation (or lack thereof of a smart valve pinch regulating mechanism: in one mechanical instance of operation, the smart valve would simply provide a visual or audio status indicator approving/disapproving the manual operation (opening, closing, modulating) of the valve regulating mechanism; in another mechanical instance of operation, the smart valve could physically unlock a latch mechanism that would permit the manual operation (opening, closing, modulating) of the valve regulating mechanism; finally, in an electromechanical instance of operation, the smart valve could physically unlock a latch mechanism and provide the actual power assisted means (e.g., electromotive force) that would automatically modulate (drive) the valve regulating mechanism (plunger) such as a gate, flap, rod, cylinder, or ball.

Most Smart valve-types can be divided into three general groups which similarly reflect the more conventional valve-type classifications: Stop valves; Check valves; and, Specialty valves. What they all have in common is a mechanism for gating flow—either with on-off or throttling operation. Stop valves are used to regulate, or in some instances, block or shut-off the flow entirely, whereas Check valves are designed to permit variable flow, albeit only in one predetermined direction at a time. Specialty valves are designated for those applications which require special purpose functionality, specification, or standards. In this case, they may necessitate special material requirements (for pressure, temperature, corrosion or erosion resistance), maintenance and repair requirements, actuation requirements, and operations requirements.

There are several classifications under the most common of the general smart valve-type categories: Multi-turn or Linear-motion valves; Quarter-turn or Rotary valves; Self-actuated valves; Control valves; and, Specialty valves. Multi-turn or Linear-motion valves consist of Gate, Globe, Pinch, Diaphragm, and Needle valves; Quarter-turn or Rotary valves consist of Plug, Ball, and Butterfly valves; Self-actuated valves consist of Check/Stop and Pressure Relief valves; Control valves consist of those which generally operate with a high degree of precision, action and reaction time; and, Specialty valves (as described above) consist of those which require special purpose operational and deployment considerations.

In general a control valve is designed to ensure the accurate proportioning or control of flow. It automatically varies the rate of flow based on signals it receives from sensing devices in a continuous process. Some valves are designed specifically as control valves. However, most types of valves can be converted to control valves (either with linear or rotary motion depending on the type of valve to be exploited) by the addition of power actuators, positioners, and/or other accessories or sensors. In this manner, the RFID enabled smart valves disclosed here can also be extended to enhance its capabilities in a similar fashion.

Within the broad range of smart valve-types are those whose identifying difference is in their actual method of actuation. These methods of actuation also determine several smart valve-type manifestations: Mechanical valves, using wheels, gears, levers, pulleys, linkages, springs, threaded shafts, and the like; Solenoid valves, which include Electromechanical, Pneumatic, or Hydraulic actuators; and, Electronic or Electric valves, which activate according to digital electronics or electrical circuits for high precision and fast reaction time.

Several smart valve-type incarnations may incorporate the hybridization of different technologies to be engineered (for automatic or reactive control of flow)—serving to respond to different environmental or conditional variants. Such variants may include temperature, pressure, flow rate, viscosity, humidity, chemistry, Ph, etc., or any combination thereof.

Wherever or whenever a means of actuation is required to facilitate a valve flow regulating “drive” mechanism (in machine coupled operation) manual or automatic actuators can be deployed. Manual actuation employs shafts, levers, wheels, and gears, for hand methods of operation to facilitate movement, while automatic actuation is ideal for those applications requiring remote operation and/or larger horse power (or torque) demands. For this reason, a separate external power source is required. For instance, this may include electromechanical drives or motors powered by electricity, and/or hydraulic or pneumatic drives powered by gas (air, nitrogen, or carbon dioxide, etc.) or fluid (oil, etc.) pressure.

The actual smart valve physical deployment can manifest itself in a variety of ways—although not limited to the following: a valve (variable clamp) that is in effect “clamped” or fastened over a deformable passageway such as a line, channel, conduit, pipeline, hose, or chute; a valve (variable clamp) whereby a deformable passageway such as a line, channel, conduit, pipeline, hose, or chute, is inserted through the apparatus itself; or, a valve whose entry/evacuation ports are fastened to in-line breakaway passageways such as a lines, channels, conduits, pipelines, hoses, or even chutes.

The primary function of the smart valve would serve to enable or preclude the flow of a liquid, vapor, gas, slurry, or dry material (powder) through a passageway. However, in the field, under temporal command and control, a smart valve would provide for the means of identification of the valve itself, an indication of the actual state of constriction—or variations thereof, and the intended position or state of the valve regulating mechanism (opened, closed, or modulated variation therein) to be either manually or automatically accommodated. With an automatic capability, the smart valve would “automatically” serve to modulate the flow by way of a powered/energized valve regulating mechanism or actuator, thus controlling the direction, rate, volume, and/or pressure, of flow through a passageway.

In general the smart valve could include one or more of the following features, although not limited to these: an RFID tag or device for the identification of the smart valve; a microcontroller/computer with or without auxiliary radio frequency (RF) communication; an electromechanical or mechanical flow constricting (limiting) mechanism; and/or, a disposable or rechargeable detached or attached power source to facilitate operation and communication in the event the power were not provided by the RFID interrogator. Instances of these components and others are illustrated in several smart valve variants in the attached drawings.

The smart valve could be used for a myriad of applications where controlling a flow would be useful. The smart valve could also be enabled by a limited number of persons on an access control list preventing inadvertent, erroneous, or malicious operation of the valve by unauthorized personnel. This access control list would be moderated, controlled, and maintained by an overseeing information management and records system.

In the event of device failure or tampering, the smart valve will recognize the state or instance of anomaly using onboard sensors and logic and initiate a failsafe protocol to circumvent dangerous administration or utilization.

It is envisaged that the valve operator at the point of dissemination would have an RFID reader with a personal digital assistant (PDA) or other portable or mobile wired/wireless hand held computer (integrated or stand alone) capable of communicating with the smart valve and concurrently capable of communicating with the main plant information management or records system. This later communication could be provided through a wireless or wired network, intranet, Internet, cellular or telephone system. As previously mentioned, with respect to the hand held computer, the RFID reader could be attached, built in, or detachable, with wired or wireless communication or stand alone capability.

The protocol of operation calls for the valve operator to interrogate the RFID tag or smart valve, and upon corroboration (matching the correct identity of the valve with the desired actuation state) the smart valve could be electromechanically unlocked (or unlatched) and the valve mechanism enabled or prepared for actuation. (The process of authentication can include a biometric interface to further corroborate operator access, permissions, and authority.)

If the smart valve is manual in nature (mechanical instance) it is left to the operator to perform the actual valve function using the built in or keyed manual actuator. On the other hand, if the smart valve is automatic in nature (electromechanical instance) the actual valve function is performed automatically without operator intervention or assistance. The process described above can also provide services including time-stamps, sensor information acquisition, and operational data collection—that may optionally be logged back to the main plant information management records system.

A failsafe protocol can also be affected within the scope of the present method and system. In the event of smart valve device failure, the operator has prior knowledge and procedures for overriding the malfunctioning smart valve without compromising intended purpose, operation, and functionality. The smart valve performs self test diagnostics periodically, upon power up, or query. In this manner, any anomaly will be reported to the operator for override, repair, or intervention. The onboard intelligence of the smart valve itself will provide an audio or visual warning, or through wireless communication, provide a warning to the hand held computer notifying the operator of a hardware failure. The operator may now invoke an override procedure to manually bypass the failure and continue with intended valve function in compliance with failsafe protocol. The overall operation is not compromised since the failure mode has been recognized and recorded, thus allowing steps to be undertaken to repair or replace the defective smart valve. This process is overseen by the information management and compliance system to conform to standards for minimizing the presence of a defective smart valve in the field. The compliance conforms to requirements within each field of application and deployment. The purpose is to safeguard against erroneous, unauthorized, malicious, or inadvertent (accidental) use, and moderate the handling, management, or replacement, of defective or obsolete devices. The actual override procedure is performed by an “authorized operator” utilizing a manual actuator or override key. The override key could be equipped with sufficient electronics (RFID tag with microelectronic interface, position Resolver, visual and/or audible indicator, and power supply) along with a physical interface to obtain position or state information (status) from the defective smart valve. Moreover, the key should be capable of setting or resetting the defective smart valve to a new state or operational position. The information gathered from the override key would be accessible via a read operation performed on the override key by an interrogating reader. This operation could also be monitored and recorded by the smart valve depending on its still remaining functional capabilities. All available information gathered via the handheld computer can be communicated to the information management and compliance system for real time feedback/notification and/or post incident investigation and analysis.

The overall process of recording these (and previously described) operations is analogous to the data logging employed in the aviation industry utilizing a “Black Box.” Other types of failure modes can be handled in a similar fashion under the guidance and instruction from the information management and compliance system. Such transactions (device status, time stamps, authorization, operations, etc.) and data storage can be made cryptographically secure preventing alteration or modification.

The Smart Valve operational components may require sterilization for safe reusability. Proper handling and disposal protocol (i.e., Bio-hazard compliance) may require that a Disposal Reader log the RFID tag of the smart valve and time-stamp the disposal, sending this information back to the main information management or records system. Even the reconstitution of the smart valve for re-use may require the time-stamping in its preparation prior to redistribution. A secure tamper-proof inlay indicator may be affixed (if required) at this point of preparation.

The attached drawings illustrate various instances of the smart valve and modes of operation. The drawings encompass several valve pinch-off regulating mechanisms and housing embodiments: multi-port stop-cock or cylinder-type (drawings 17 and 18) with 2 and 3 ports, and their corresponding flow channels (drawings 19 and 20), respectively, with a rotational shaft; butterfly-type, with a rotational shaft (drawing 21); and, gate, globe, or needle-type, with a screw shaft (drawings 22 and 23).

These smart valves contain lockable-latch mechanisms (but could simply contain an insecure latch) with valve regulating mechanisms that are either manual or automatic in function. As illustrated, the instances of manual smart valves have manually operated actuators which serve to activate the valve pinch-off regulating mechanism. On the other hand, the automatic smart valves have an automatic (electromechanical) valve pinch-off regulating mechanism to serve the same functionality.

In the series of Figures numbered 17, 18, 21, and 23, there is illustrated RFID enabled smart valves with electromotive actuators. An electromotive actuator has an electric motor drive that provides torque to operate a valve pinch-off regulating mechanism. These actuators are frequently used on multi-turn valves such as gate or globe valves. With the addition of a gearbox, they can be utilized on plug, butterfly, or other quarter-turn valves. Other automatic actuators considered for use in the smart valve engineering can include those based on other propulsion forces such as hydraulic, pneumatic, and temperature activated shape memory alloys.

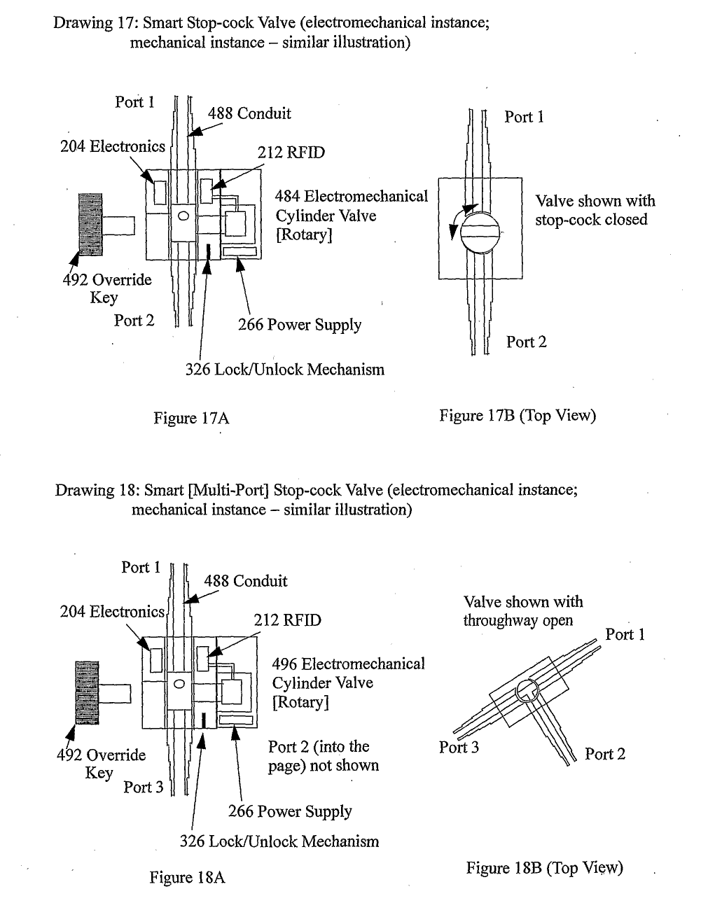

In the series of Figures numbered 17 and 18 there is illustrated smart stop-cock valves employing a cylindrical valve regulating control mechanism in multi-port configurations. The cylindrical plunger and housing accommodate a rotating cavity that allows for flow pathways in a variety of channel combinations. Such smart stop-cock valves offer a wide range of flexibility in that several port and throughway combinations are available in a compact package. For example, in FIG. 17, the open actuator position allows for a straight-through flow pathway, but shuts off flow when the cylinder is rotated 90 degrees to block the flow passage. This configuration is commonly used for on/off and throttling services. In FIG. 18, one can deduce that there are a host of flow pathway combinations to be had depending on the angular position of the shaft relative to the valve housing. A cylindrical plunger with a branching cavity hollow juxtaposes with the housing embodiment of the valve in a variety of positions—yielding variety of flow pathways.

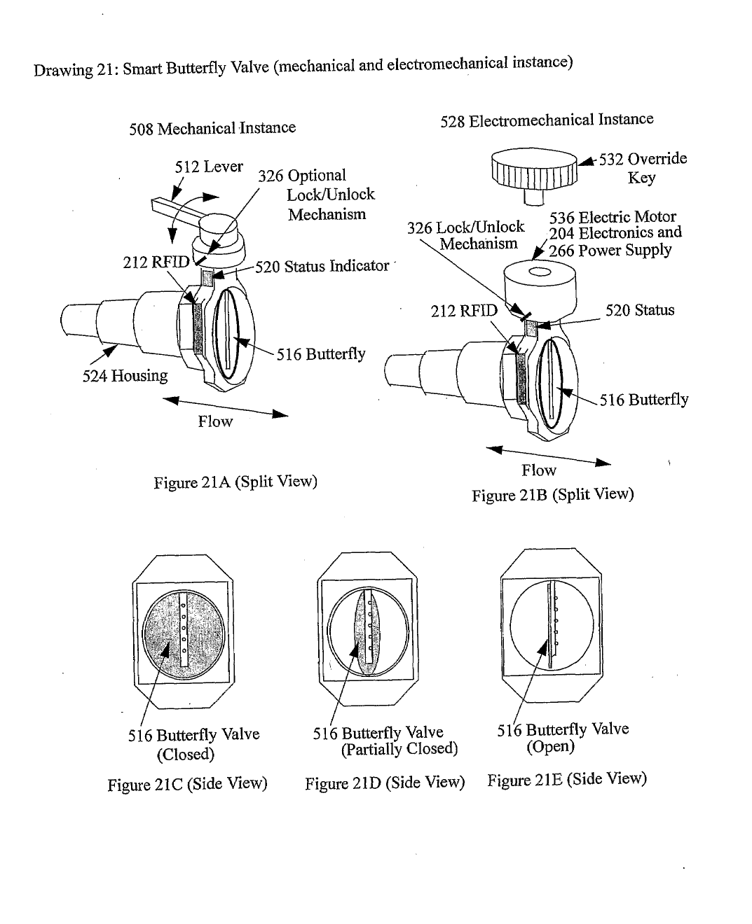

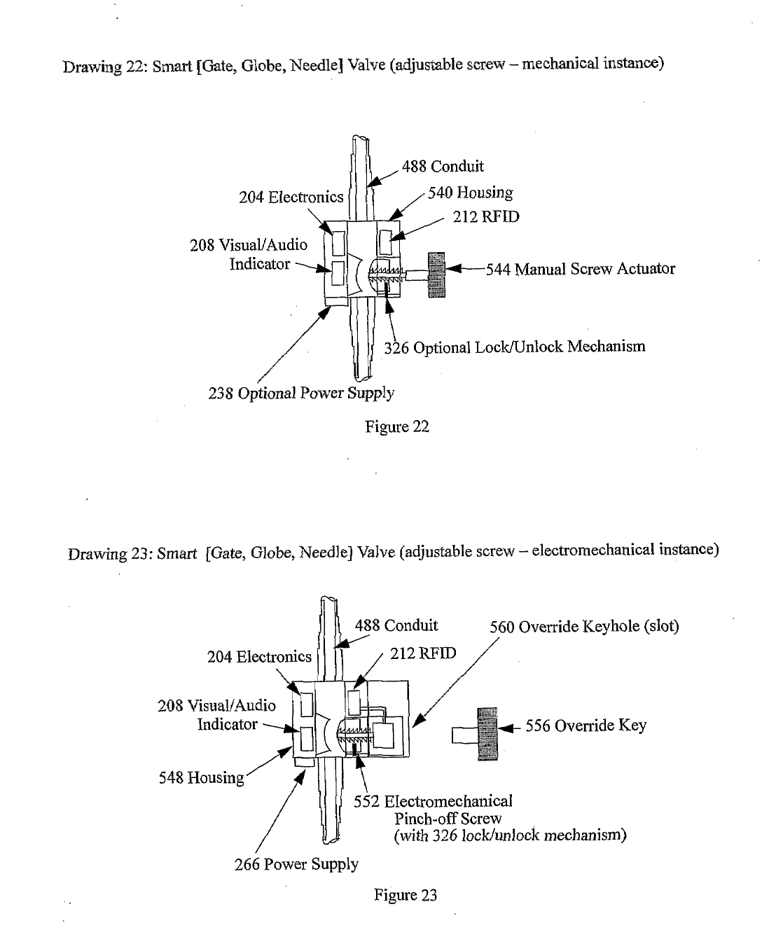

In addition to the stop-cock type smart valves shown, FIGS. 21, 22, and 23, indicate other straight-through type smart valve embodiments in the form of butterfly, gate, globe, pinch, or needle type plunger configurations. Hence it is safe to say that many purposeful RFID enabled smart valves can be derived from virtually any base valve-types in addition to those which have been described in the document.

In the series of Figures numbered 17 is a diagram illustrating a smart stop-cock valve (electromechanical instance; mechanical instance similar illustration). FIG. 17A shows the conduit 488, RFID 212, enabled lock/unlock mechanism 326, electronics 204, override key 492, power supply 266 and electromechanical cylinder rotary valve 484. An electromechanical motor/servo adjusts a valve thus unrestricting or restricting the flow in the conduit 488 when the RFID enabled lock mechanism 326 is unlocked. FIG. 17B shows a top view of the valve in the state of restricted flow.

In the series of Figures numbered 18 is a diagram illustrating a smart multi-port stop-cock valve (electromechanical instance; mechanical instance similar illustration). FIG. 18A shows the conduit 488, RFID 212, enabled lock/unlock mechanism 326, electronics 204, override key 492, power supply 266 and electromechanical cylinder rotary valve 496. An electromechanical motor/servo adjusts a valve thus unrestricting or restricting the flow in the conduit 488 when the RFID enabled lock mechanism 326 is unlocked. FIG. 18B shows a top view of the valve illustrating the multiple ports.

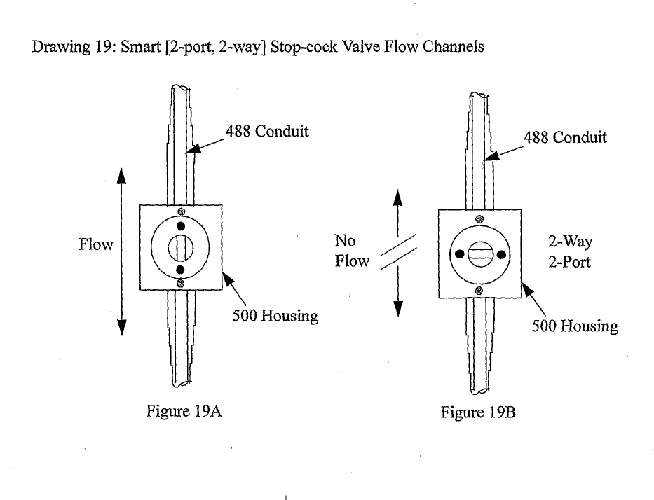

In the series of Figures numbered 19 is a diagram illustrating a smart stop-cock valve. FIG. 19A shows the conduit 488, housing 500, and alignment markers indicating the position of the valve being in an unrestricted flow state. FIG. 17B shows the conduit 488, housing 500, and alignment markers indicating the position of the valve being in a flow restricting state.

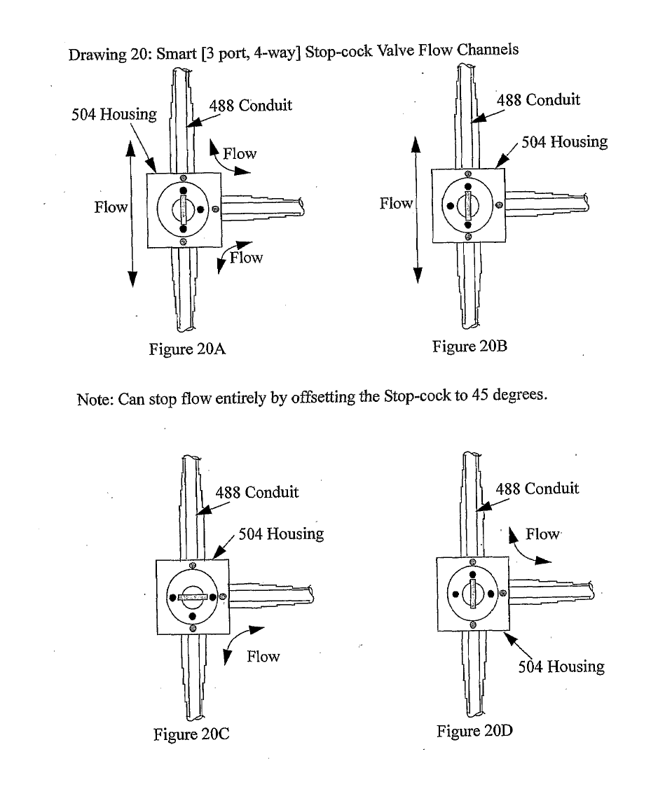

In the series of Figures numbered 20 is a diagram illustrating a smart 3 port 4 way stop-cock valve. FIG. 20A shows the conduit 488, housing 500, and alignment markers indicating the position of the valve being in an unrestricted flow state for all 3 ports. FIG. 20B shows the conduit 488, housing 500, and alignment markers indicating the position of the valve being in a straight through flow state. FIG. 20C shows the conduit 488, housing 500, and alignment markers indicating the position of the valve allowing flow through the lower two conduits. FIG. 20D shows the conduit 488, housing 500, and alignment markers indicating the position of the valve allowing flow through the top two conduits. As noted the valve can also be positioned to prevent flow in all 3 conduits.

In the series of Figures numbered 21 is a diagram illustrating a smart butterfly valve (mechanical 508 and electromechanical instance 528). FIG. 21A shows the housing 524, RFID 212, status indicator 520, optional lock/unlock mechanism 326, a lever 512 affecting the butterfly 516 valve. FIG. 21B shows the housing 524, RFID 212, status indicator 520, lock/unlock mechanism 326, power supply 266, electric motor 536 affecting the butterfly 516 valve, and an override key 532. FIGS. 21C, 21D, and 21E, illustrates side views of the butterfly valve in the closed, partially closed, and open states.

In the series of Figures numbered 22 is a diagram illustrating smart [Gate, Globe, Needle] valve (adjustable screw mechanical instance). Illustrated is the conduit 488, housing 540, RFID 212, optional lock/unlock mechanism 326, visual/audio indicator 208, optional power supply 238, associated electronics 204, and a manual screw actuator 544. When the RFID 212 enables the indicator 208 or lock mechanism 326 the manual screw actuator 544 can be used to restrict or open the flow.

In the series of Figures numbered 23 is a diagram illustrating a smart [Gate, Globe, Needle] valve (adjustable screw electromechanical instance). Illustrated is the conduit 488, housing 548, RFID 212, lock/unlock mechanism 326, visual/audio indicator, associated electronics 204, power supply 266, and an electromechanical pinch-off screw actuator 522. When the RFID 212 enables the indicator 208 and lock mechanism 326 the electromechanical pinch-off screw actuator 552 can be used to restrict or open the flow. An override key is shown as 556.

### Smart Syringe for Medical Applications

A “conventional” syringe is a manual or automatic (power assisted) injection-mechanical device used to transfer a fluid preparation or therapy (liquid or gas) from a reservoir through a discharge channel via a nozzle in a controlled and accurate manner. Some of the fluid discharge or transfer control variables include the direction, volume, rate, and/or pressure of flow.

The following “Smart Syringe” description envelopes this basic syringe principal however offers much greater capability and purpose by including a Radio Frequency Identification (RFID) tagging device and interface with bi-directional communication. The invention itself has a provision for discharge (or a means of fluid transfer), and hence delivery, through a nozzle and channel (coupler and/or tubing) and/or a hollow needle accessory or attachment for penetration directly into a host or an Intravenous set Y-connector or the like. Its purpose is for improving the preparation, transport, delivery, administration, and disposal (e.g., for enhanced patient safety and quality of care) of an injectable or oral fluid preparation or therapy (including chemical, medication, drug, infusion fluid, vaccine, serum, or vitamin).

For the purpose of demonstrating the invention of a Smart Syringe, the emphasis (as depicted here) is on a medical setting and application environment. This serves to demonstrate the design, operation, and functionality of a Smart Syringe, however, it is understood that it can be successfully applied to other application areas including industrial and commercial usage along these lines.

The RFID device of the syringe can be either passive or active. It has to have the added capability of setting or releasing the latch mechanism of the syringe. It is the RFID or an interfaced or integrated module with associated electronics that enables or disables the latch/lock upon confirmation between the RFID of the smart syringe (prescription), care provider, patient, and perhaps knowledge from an overseeing information management system. The power for the module (if required) could come from the RFID device directly or from its own on-board power supply.

The smart syringe with its RFID enable mechanism would encompass one or more of the following items: a status indicator; a shut-off mechanism or latch/lock that would be included—capable of being activated or de-activated by the care provider upon correct correspondence between the RFID tags of the smart syringe, patient, and care provider; a mechanism for depressing/expanding the syringe plunger—allowing manual care provider mode of operation; an electromechanical actuator for the automatic injection or depression/expansion of the syringe plunger using a motor or servo or the like—allowing automatic care provider mode of operation; a mechanism that detects plunger position—recording the amount of fluid dispensed—to be interrogated by the RFID reader/mobile or portable computing device. (This could incorporate a “resolver” such as a diffraction grating/Light Emitting Diode and photo-detector combination, linear-variable potentiometer, gear-transmission based rotary-variable potentiometer, etc.)

It should be noted that in a physical realization instance, the latch/lock device itself could also be an adaptor retrofitted to existing syringes as a collar device that could also be reusable and even re-sterilizable. This device would again contain an RFID tag for identification as well as control (including actuation). The lock or latch mechanism would securely grip the syringe plunger preventing administration of contents until its RFID was read and corroborated with the care provider, patient—at which point the release mechanism would be enabled thereby freeing the plunger to allow authorized administration the fluid.

It should be noted that in some instances such as administering/injecting a medication through a smart syringe (within the near field of the RFID Reader 116), several RFID tags may have to read at the same time in order for the syringe latch or valve to allow for the flow of medication contained in the syringe into the intravenous (IV) set. In this case, say a Y connector (with accompanying injection site), or some other place or point of ingress within the channel, it could have an RFID tag affixed to it to be read at the same time as the smart syringe RFID tag. By the correct positioning of the handheld RFID Reader (in order to corroborate the compliance and warrant the opening of the latch or valve on the syringe), the medication can be permitted to flow through/into the Y-connector of the Intravenous set, and on into the patient.

If the amount of dispensed quantity actually differs from that of the quantity specified by the overseeing physician or clinician, the shut-off device could be activated to cut off the fluid flow or supply, respectively. Contrastively, this mechanism can also be adapted to dispense the correct amount of injectable fluid.

Upon dispensing of the drug from the syringe, the smart syringe RFID would again be read at disposal. More specifically, when the syringe is disposed of after use it is typically disposed of securely. An RFID reader located on a disposal chute would read the RFID, time-stamp the event, and provide this information back to a central main patient data base and records system. The timestamp information can also be used to close the loop on when, where, to whom, by whom, and what fluid was administered. It is apparent that the reading of the smart syringe ID is useful even if the syringe RFID device is only used for identification.

It is envisaged that the clamp operator at the point of actuation would have an RFID reader with a personal digital assistant (PDA) or other portable or mobile wired/wireless hand held computer (integrated or stand alone) capable of communicating with the smart clamp and concurrently capable of communicating with the main plant information management system. This later communication could be provided through a wireless or wired network, intranet, Internet, cellular or telephone system. As previously mentioned, with respect to the hand held computer, the RFID reader could be attached, built in, or detachable, with wired or wireless communication or stand alone capability. The protocol of operation calls for the clamp operator to interrogate the RFID tag or smart clamp and upon corroboration (matching the correct identity of the clamp with the desired actuation state) the smart clamp could be electromechanically unlocked (or unlatched) and the pinch mechanism enabled or prepared for actuation. If the smart clamp is manual in nature (mechanical instance) it is left to the operator to perform the actual pinch-off function using the built in or keyed manual actuator. On the other hand, if the smart clamp is automatic in nature (electromechanical instance) the actual pinch-off function is performed automatically without operator intervention or assistance. The process described above can also provide services including time-stamps, data collection, and sensor information that may optionally be logged back to the main plant records system.

A failsafe protocol can also be affected within the scope of the present method and system. In the event of smart clamp device failure, the operator has prior knowledge and procedures for overriding the malfunctioning smart clamp without compromising intended purpose, operation, and functionality. The smart clamp performs self test diagnostics periodically, upon power up, or query. In this manner, any anomaly will be reported to the operator for override, repair, or intervention. The onboard intelligence of the smart clamp itself will provide an audio or visual warning, or through wireless communication, provide a warning to the hand held computer notifying the operator of a hardware failure. The operator may now invoke an override procedure to manually bypass the failure and continue with intended clamp function in compliance with failsafe protocol. The overall operation is not compromised since the failure mode has been recognized and recorded, thus allowing steps to be undertaken to repair or replace the defective smart clamp. This process is overseen by the information management and compliance system to conform to standards for minimizing the presence of a defective smart clamp in the field. The compliance conforms to requirements within each field of application and deployment. The purpose is to safeguard against erroneous, unauthorized, malicious, or accidental use, and moderate the handling or management of defective or obsolete devices. The actual override procedure is performed by an authorized operator utilizing a manual actuator or override key. This operation is also monitored and recorded by the smart clamp and communicated via the handheld computer to the information management and compliance system.

Other types of failure modes would be handled in a similar fashion under the guidance and instruction from the information management and compliance system.

The Smart Syringe operational components may require sterilization for safe reusability. Proper handling and disposal protocol (i.e., Bio-hazard compliance) may require that a Disposal Reader log the RFID tag of the smart syringe and time-stamp the disposal, sending this information back to the main information management or records system. Even the reconstitution of the smart syringe for re-use may require the time-stamping in its preparation prior to redistribution. A secure tamper-proof inlay indicator may be affixed (if required) at this point of preparation.

The attached drawings illustrate various instances of the smart syringe and modes of operation. The drawings encompass several syringe/plunger regulating mechanisms and housing embodiments.

These smart syringes contain lockable-latch mechanisms (but could simply contain an insecure latch) with control mechanisms that are either manual or automatic in function. As illustrated, the instances of manual smart syringes have manually operated actuators which serve to activate or unlock the syringe control mechanism. On the other hand, the automatic smart syringes have an automatic (electromechanical) mechanism to serve the same functionality.

In the series of Figures numbered 24 there is illustrated the basic smart syringe with an RFID tag and control unit or valve located at or near the nozzle. The control mechanism prevents the depression of the thumb flange indirectly by the control valve not allowing the contents of the syringe from being transferred and therefore dispensed.

In the series of Figures numbered 25 there is illustrated a latching/locking functionality with the RFID and control mechanism located at the finger-flange base of the syringe. The control mechanism prevents depression of the thumb flange directly by using a friction grip or keyed stop actuator.

In the series of Figures numbered 26 there is illustrated the smart syringe with a simple operator responsible “go/no-go” indicator. (This implementation is among the most vulnerable of the smart syringes due to fact it does not contain a secured latch/lock.)

In the series of Figures numbered 27 there is illustrated two manual implementation embodiments using a rotational and push-pull latch.

In the series of Figures numbered 28 there is illustrated two implementation scenarios where the RFID tag and control are affixed to the syringe: in either a clam-shell arrangement, or removable thumb-flange (allowing access to the plunger shaft) whereby the RFID and control unit could be slid over and along the plunger shaft to the finger flange for securing.

In the series of Figures numbered 29 there is illustrated how the concept can be extended to legacy syringes, whereby a syringe access capsule/enclosure is RFID controlled and enabled prevents miss-use of the legacy syringe.

In the series of Figures numbered 30 there is illustrated a new syringe design where the plunger shaft is modified to incorporate a collapsible latch mechanism, such that it can not be activated for use until corroborated by an interrogating reader.

In the series of Figures numbered 31 there is illustrated possible position (resolver) sensors that could be incorporated into the basic smart syringe allowing for the position of the plunger itself to be an observable/control parameter.

In the series of Figures numbered 32 there is illustrated in more detail possible thumb-rest removal and attachment mechanisms for the purpose of providing better access (both ingress and egress) to the RFID control assembly. In doing so, enhancements in both simplicity and ergonomics in design are realized.

In the series of Figures numbered 33 there is illustrated an intersticed design implantation where the RFID with control (valve or pinch mechanism) is intersticed between the needle and syringe.

In the series of Figures numbered 34 there is illustrated a motorized or automatic discharge implementation embodiment using RFID with control and actuation.

In the series of Figures numbered 35 there is illustrated another RFID with control mechanism embodiment with a modified cylindrical plunger.

In the series of Figures numbered 24 is a diagram illustrating a smart syringe: RFID with control mechanism at nozzle. FIG. 24A illustrates a typical syringe. FIG. 24B illustrates the syringe modified to include a position sensor 592, an RFID 212, an optional indicator 596, control line 600, and a control valve 604. The RFID 212 would enable the control valve 604 allowing the syringe to be discharged.

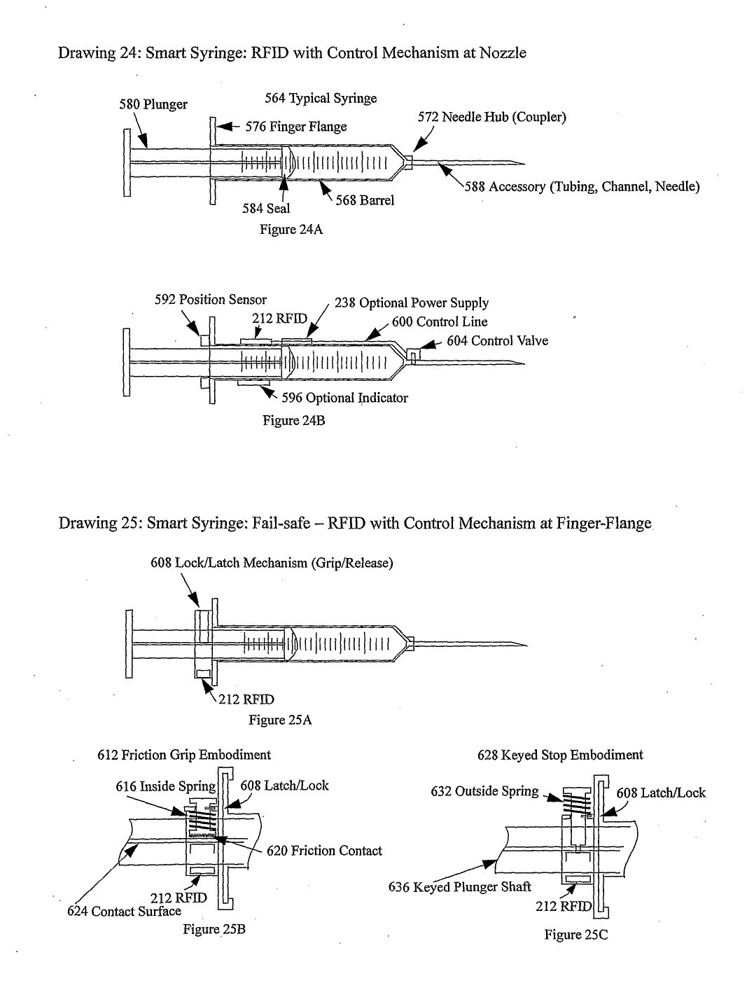

In the series of Figures numbered 25 is a diagram illustrating a smart syringe: fail-safe, RFID with control mechanism at finger flange. FIG. 25A illustrates a typical syringe incorporating RFID 212 and Lock/Latch Mechanism 608 grip release. FIG. 25B illustrates the syringe modified to include a friction grip embodiment 612. The RFID 212 enables the lock/latch 608 releasing the spring 616 thus releasing the friction contact 620 from the plunger contact surface 624 allowing a person to discharge the content of the syringe. FIG. 25C illustrates the syringe modified to include a keyed stop embodiment 628. The RFID 212 enables the lock/latch 608 releasing the spring 632 thus releasing the key from the keyed plunger surface 636 allowing a person to discharge the content of the syringe.

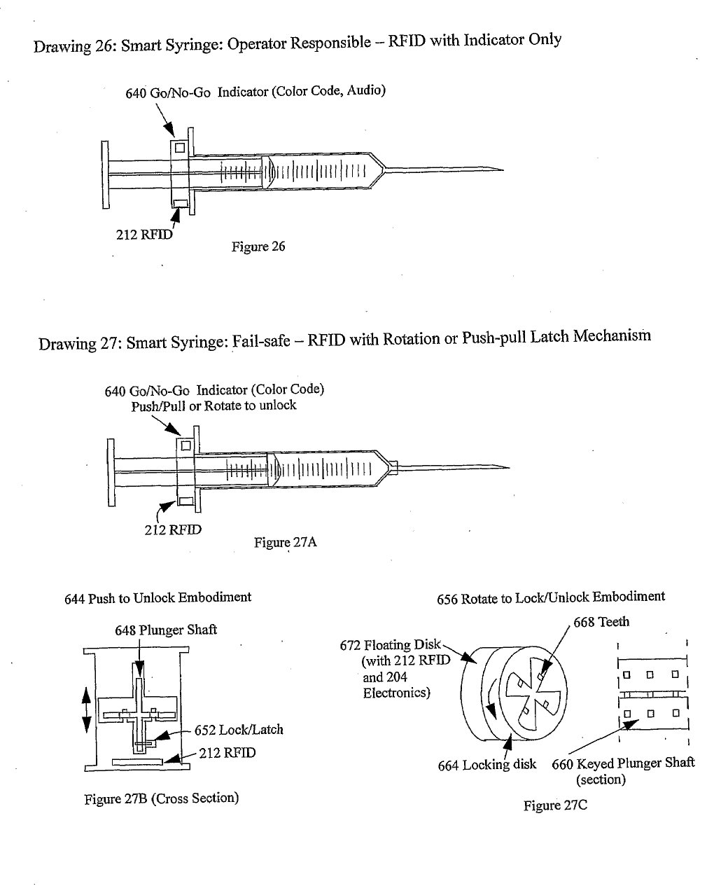

In the series of Figures numbered 26 is a diagram illustrating a smart syringe: operator responsible RFID with indicator only). FIG. 26 illustrates the syringe embodiment with RFID 212 and a go/no-go indicator, indicating that a person may discharge the content of the syringe.

In the series of Figures numbered 27 is a diagram illustrating a smart syringe: fail-safe, RFID with rotation or push-pull latch mechanism at finger flange. FIG. 27A illustrates a typical syringe incorporating RFID 212 and go/no-go indicator 640 with a rotate or push-pull mechanism to unlock the syringe. FIG. 27B illustrates the push to unlock embodiment 644. The RFID 212 enables the indicator 640 indicating that the restricting mechanism (key in hole) can be released from the keyed plunger shaft surface 648 allowing a person to discharge the content of the syringe. FIG. 27C illustrates the rotate to lock/unlock embodiment 656. The RFID 212 and associated electronics are enclosed on a floating disk 672 enables the indicator 640 indicating that the restricting mechanism (key in hole) on the locking disk 664 can be released from the keyed plunger shaft surface 660 allowing a person to discharge the content of the syringe. 668 illustrate the teeth on the locking disk and the corresponding holes on the keyed plunger shaft 660.

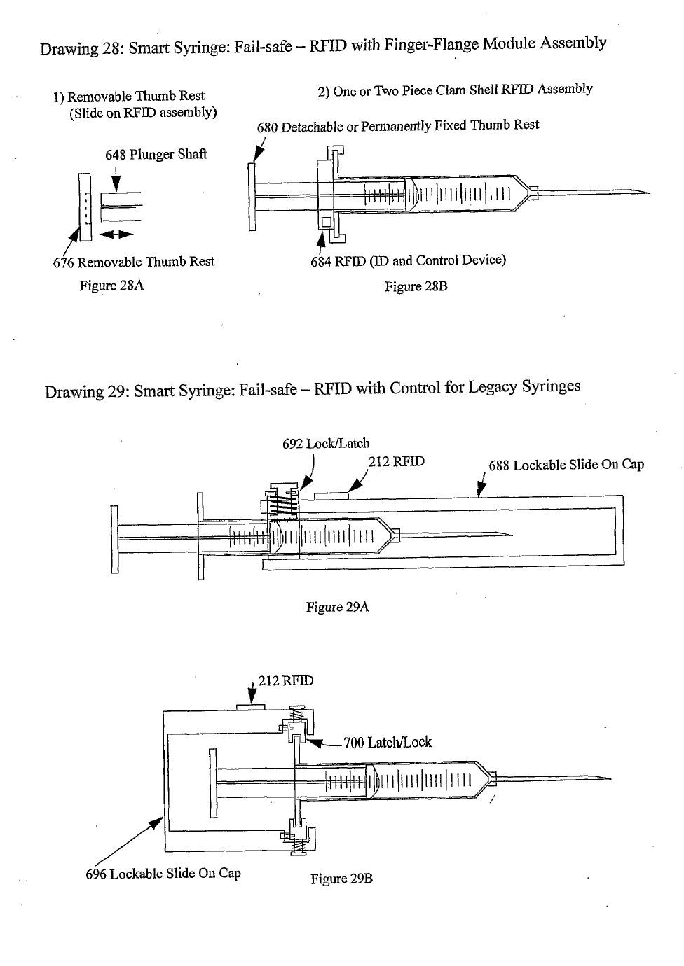

In the series of Figures numbered 28 is a diagram illustrating a smart syringe: fail-safe, RFID with finger-flange module assembly. FIG. 28A illustrates a syringe incorporating a removable thumb rest 676 attachable to the syringe plunger shaft 648 thus allowing the sliding on of the RFID 684 ID and control device. FIG. 28B illustrates the detachable thumb rest 680 for the case of the slide on RFID unit 684 or the permanently fixed thumb rest 680 for the case of the clam shell RFID assembly 684

In the series of Figures numbered 29 is a diagram illustrating a smart syringe: fail-safe, RFID with control for legacy syringes. FIG. 29A illustrates a syringe incorporating a lockable slide on cap 688, RFID 212, and lock/latch mechanism 692. In this manner a typical syringe would be housed within locked slide on cap preventing the use of the syringe unless enabled by the RFID 212 and lock/latch mechanism 692. FIG. 29B illustrates an embodiment with the slide on cap encasing the syringe plunger. In this manner a typical syringe (plunger) would be housed within locked slide on cap 696 preventing the use of the syringe unless enabled by the RFID 212 and lock/latch mechanism 700.

In the series of Figures numbered 30 is a diagram illustrating a smart syringe: fail-safe, RFID with a collapsible latch mechanism. FIG. 30A illustrates a syringe incorporating an electromechanical lock 704, RFID 212, and cap 708. In this manner a modified syringe plunger would be housed within a cap preventing the use of the syringe unless enabled by the RFID 212 and electromechanical lock 704. The cap 708 can be withdrawn as shown in FIG. 30B with lobe 716 and seat 720 responsible for retaining the cap 708 in the withdrawn position attached to the modified syringe plunger. In this manner a syringe (plunger) would be housed within locked cap 708 preventing the use of the syringe unless enabled by the RFID 212 and lock/latch mechanism 704. Once the lock is released and the cap pulled back and affixed to the plunger the syringe can be discharged. The cap can be withdrawn either by pulling it back one the lock is released or rotated and pulled back.

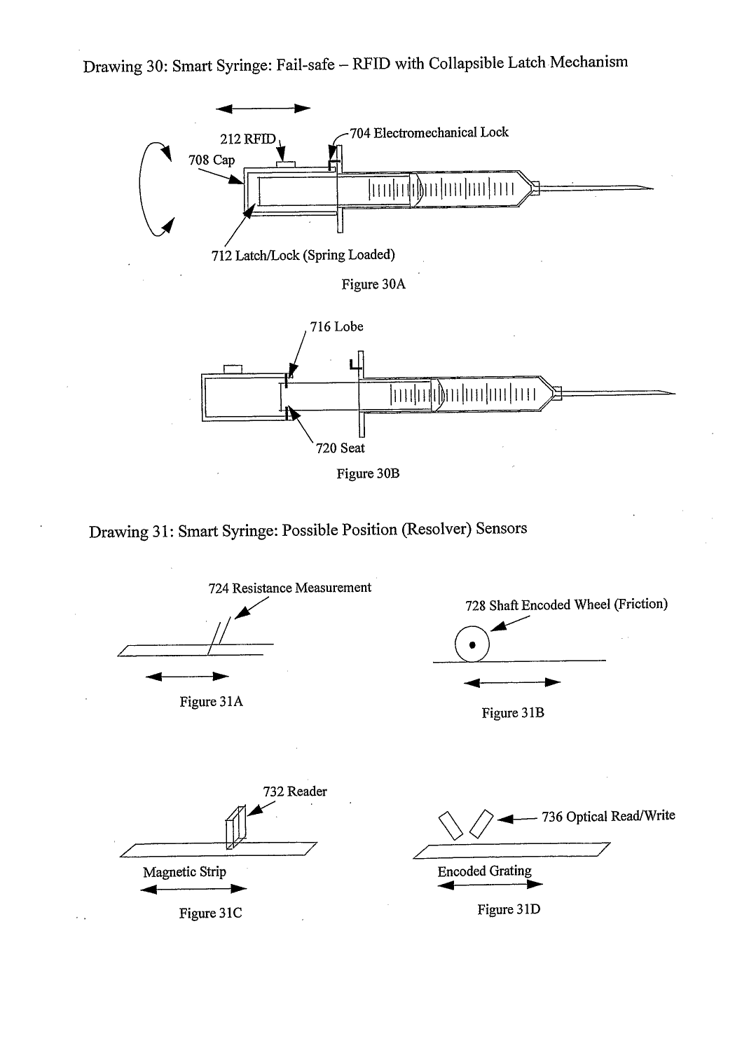

In the series of Figures numbered 31 is a diagram illustrating possible position resolving sensors. FIG. 31A illustrates a means of detecting position via resistance 724. FIG. 31B illustrates a means of detecting position via shaft encoded friction wheel 728. FIG. 31C illustrates a means of detecting position, via a magnetic strip reader 732, reading position information from a magnetic strip. FIG. 31D illustrates a means of detecting position via an optical reader 736 reading position from an encoded grating.

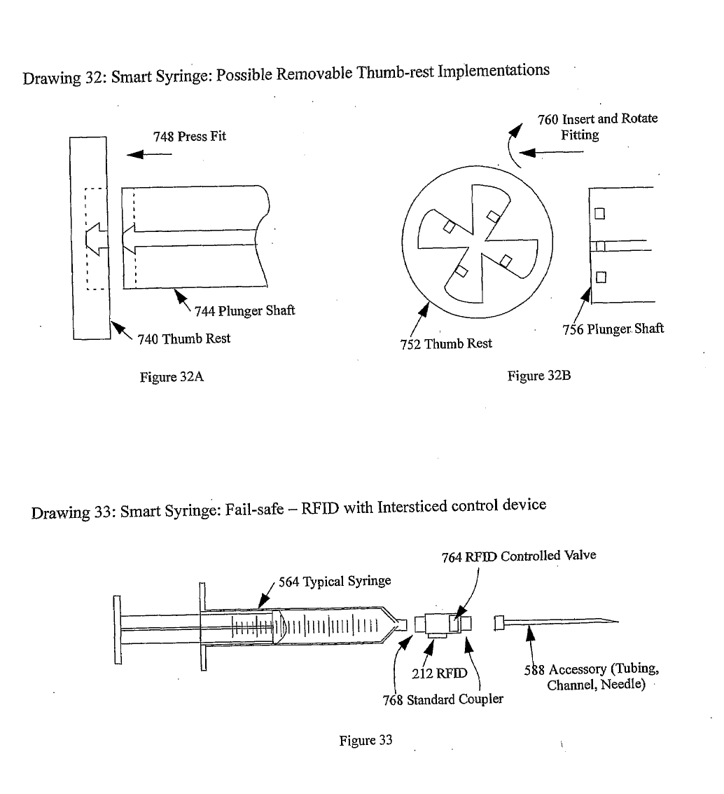

In the series of Figures numbered 32 is a diagram illustrating possible removable thumb-rest implementations. FIG. 32A illustrates a press fit embodiment 748 where the plunger shaft 744 is pressed into the thumb rest 740. FIG. 32B illustrates an insert and rotate embodiment 760 where the plunger shaft 756 is fit into the thumb rest 752. The thumb rest is retained in position by a key and slot mechanism.

In the series of Figures numbered 33 is a diagram illustrating a smart syringe: fail-safe RFID with Intersticed control device 764. FIG. 33 illustrates the typical syringe 564 that can be coupled via a standard couple 768 to the RFID control valve inclusive of an RFID 212 and flow control mechanism. The RFID control valve is also capable of being coupled to the syringe accessory 588 (needle, tube, channel, etc.)

In the series of Figures numbered 34 is a diagram illustrating a smart syringe: fail-safe RFID with motorized control and actuator device. FIG. 34A illustrates the typical syringe 564 that can be fitted with a RFID controlled actuator motorized plunger 772. The RFID controlled actuator motorized plunger 772 is controlled by a motor 776 activated by the RFID 212. FIG. 34B illustrates the motor, plunger shaft, and linear actuation possible within the present embodiment.

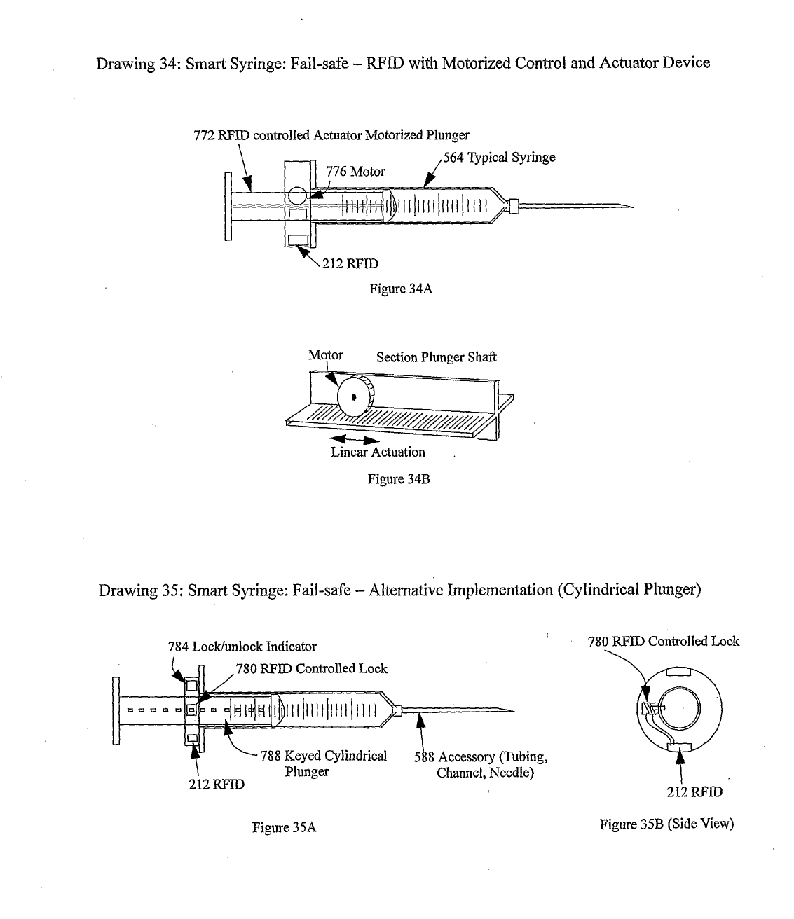

In the series of Figures numbered 35 is a diagram illustrating a smart syringe: fail-safe RFID with alternative implementation (keyed cylindrical plunger 788). FIG. 35A illustrates the typical syringe that can be fitted with a RFID 212 and lock/unlock indicator 784 and a RFID controlled lock 780. FIG. 35B illustrates the RFID controlled lock 780 and the RFID from an alternative view. 588 illustrates various attachments associated with a syringe.

### Smart Coupler for Medical Applications

A “conventional” coupler is a manually operated mechanical device used to securely interface and typically connect to another coupler (i.e., female to male coupling) to create a passageway to allow for the flow of a liquid, vapor, gas, slurry, or dry material (powder), through two connected lines, channels, conduits, pipelines, or hoses.

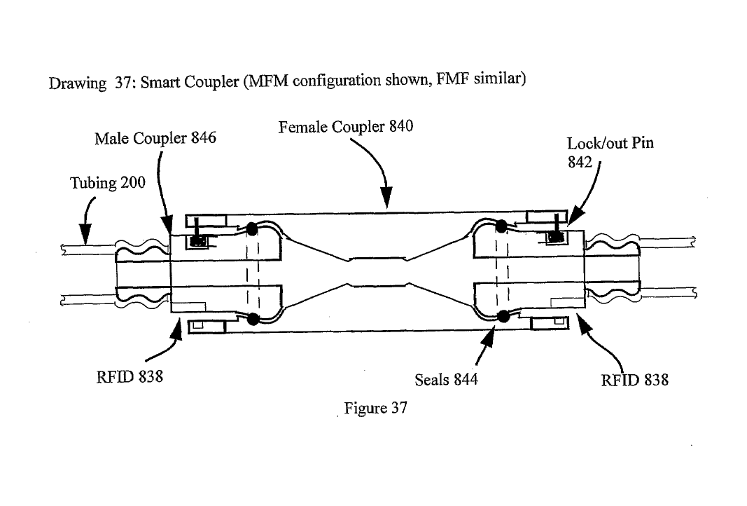

The following “Smart Coupler” illustrated in FIG. 37 description envelops this basic coupler principle, however offers much greater capability and purpose by including a Radio Frequency Identification (RFID) tagging 838 device and interface with bi-directional communication. A smart coupler 846 can encompass several connect/disconnect embodiments. The smart coupler invention described here incorporates an RFID tag 838 and accompanying interface (or RFID system on a chip) (in situ and/or external) in a method and system to control the mechanical operation (e.g., user's thumb) of the act of coupling (a male to female mating). By enabling or precluding (with lock out pins 842) an instance of user operation of the coupler regulating mechanism, two separate lines 200 can be attached or removed or prevented from being attached or removed, respectively. Each smart coupler in this embodiment are identical and therefore require a mating gateway conduit or channel 840 with groves on either side to accommodate locking rings/pins of the smart coupler's thumb operated clasping actuator.

Hence, in addition to facilitating the flow by coupling two of the enabled smart couplers together, through a mating via (gateway), the coupler thumb lever mechanism incorporates a lockable-latch which will prevent unauthorized, erroneous, or inadvertent operation of the coupler.

In a medical instance, smart couplers can be used to safely and securely connect medical tubing such as intravenous lines (containing medication, testing dyes, or blood).

Much of what is discussed about the methods of deployment, regarding a smart coupler, also pertains to smart medical devices in general. There are parallels to be drawn in both methods and systems regarding RFID Enabled Requirements, Enabled Operation, Visual and Auditory Indicators, Operation and Protocol, and Recycling—Disposal, Sterilization, and Reuse.

In FIG. 37 is a diagram illustrating a smart coupling device—Male-Female-Male Instance. FIG. 37 shows the tubing 200 affixed to a male coupler 846. The male coupler 846 has an RFID tag 838 capable of controlling a lock/out pin 842 thus preventing the insertion of the male coupler 846 into the female coupler 840.

### Smart Pipette for Medical Applications

A “conventional” pipette is a manual or automatic (power assisted) injection-mechanical device used to transfer a fluid sample preparation, or therapy (liquid or gas), from a reservoir through a holding chamber 868 and discharge channel, via a nozzle in a controlled and accurate manner. Some of the fluid discharge or transfer control variables include the direction, volume, rate, and/or pressure of flow. The pipette can acquire fluid (sucking up the fluid), contain fluid, or discharge fluid (expel fluid) into or from the pipette's holding chamber.

The following “Smart Pipette” description envelopes this basic pipette principal however offers much greater capability and purpose by including a Radio Frequency Identification (RFID) Reader 850 device and interface with bi-directional communication. (It can also have its own RFID tag in some embodiments.) The smart pipettes can also communicate with a hand held PDA or mobile computer 115, or directly to an existing laboratory information and communications system, via wireless access points (e.g., 802.11x). The invention itself has a provision for discharge (or a means of fluid transfer), and hence delivery, through a nozzle and channel (coupler and/or tubing) and/or a hollow needle accessory or attachment, for penetration/delivery directly into a host of test tubes 864, petri dishes 860, laboratory slides, or other clinical or laboratory bins and containers (e.g., flasks).

A primary function of the smart pipette is to prevent errors from occurring when handling test samples. The RFID enabled lock-out thumb activated depressor 858, or fluid release depressor 872, will help prevent the operator from placing fluid in an unintended test tube, petri dish, or container, by blocking the action entirely, and even providing a visual or auditable 874 warning, from the device itself, or through a hand held PDA or mobile computer 115. Its ancillary purpose is for the monitoring and logging (time stamping) of data and general information, and testing or research processes and procedures (for later examination). Several benefits can be had in conforming to clinical and laboratory standards, protocols, and practices set out by policy makers. For instance, improvements in quality control, and efficiency, can increase productivity in the facility whilst reducing errors. Proper handling protocols in place for the handling and disposing of Bio-hazardous materials can also be improved with such a method and system. This can be achieved and affected in part by the guidance of a laboratory expert system 102b, and overseeing smart medical compliance ICT system 100b.

It should also be noted that the recording and time stamping features of such a method and system would be particularly useful in the developments of new medicines and treatments. It can record parameters such as what substances were involved in a particular experiment, when they were created, and/or when they were added or removed in any of the particular medical apparatuses. Any observations can be entered by the technician via his/her PDA device 115 so that it can be stored in a laboratory record data base. This can be useful when subsequent (even unforeseen) evaluation is required to study particular events that took place earlier. For instance, a chemical formula or recipe can be reverse engineered with the knowledge stored in the laboratory data base. Also, it can be determined when and where certain compositions, compounds, and titrations were formed and any intermediate reactions therein.

For the purpose of demonstrating the invention of a smart pipette, the emphasis (as depicted here) is on a medical, clinical, or laboratory setting, and application environment. This serves to demonstrate the design, operation, and functionality of a smart pipette, however, it is understood that it can be successfully applied to other application areas including industrial and commercial usage along these lines.

The smart pipette has an electromagnetic field extender 854 (with accompanying shield), on or near its discharge tip so that the near field communications determined by the RFID Reader 850 is very narrow. In this way, the smart pipette will only read (or sense the intended laboratory apparatus or specimen). To help facilitate and conform to this requirement, a corresponding test tube, petri-dish, sample slide, and other tagged apparatuses will all have a “narrow field” RFID tags as well.

Much of what is discussed about the methods of deployment, regarding a smart pipette, also pertains to smart medical devices in general. There are parallels to be drawn in both methods and systems regarding RFID Enabled Requirements, Enabled Operation, Visual and Auditory Indicators, Operation and Protocol, and Recycling—Disposal, Sterilization, and Reuse.

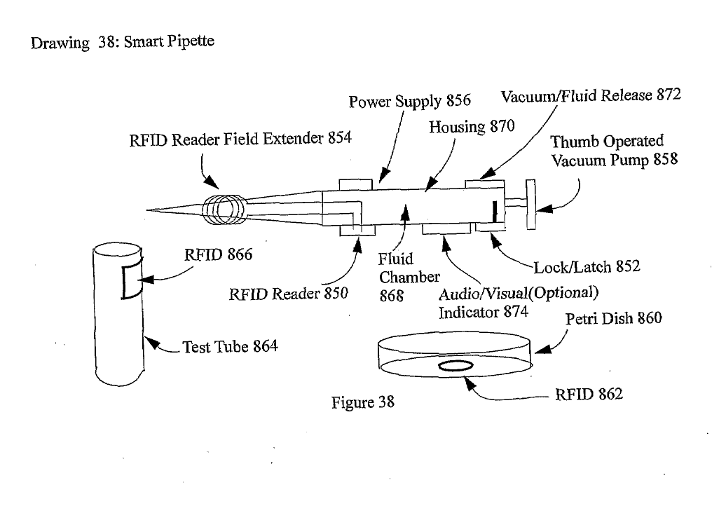

FIG. 38 is a diagram illustrating one embodiment of a smart pipette device. FIG. 38 shows a petri dish 860 with an affixed RFID tag 862 and a test tube 864 affixed with an RFID tag 866. The pipette incorporates a housing 870, a thumb operated vacuum pump 858, a vacuum release 872, power supply 856, fluid chamber 868, RFID reader 868, an audio/visual indicator 874, and an RFID field extender 854. Upon corroboration between the pipette RFID reader 850 and, for instance, the RFID tag 866 of the test tube 864, the lock or latch 852 will be released and the fluid delivered to or extracted from the test tube 864.

1. A system for providing patient care at a point of care (POC) comprising:
an RFID tag for a care provider at the POC;
an RFID tag for a patient at the POC;
an RFID Reader;
a portable hand held computer for a care provider at the POC
a medical device at the POC having an RFID tag;
the medical device having an operable element with a control device for enabling of the operable element;
and a computing system for connecting the above items such that the control device allows actuation of the operable element only in the event that the reader detects the RFID of the care provider and of the patient and of the medical device and the computing system confirms that they are properly in accordance with a prescribed medical treatment.

2. A system according to claim 1 wherein the computing system is arranged to provide a time stamp record of an actuation of the operable element.

3. A system according to claim 2 wherein the medical device includes a sensor for detecting operation and a completion of an operation and wherein the computing system is operable to record both operation and completion.

4. A system according to claim 3 wherein the computer system is arranged to provide a reminder to the portable hand held computer if not completed.

5. A system according to claim 1 wherein the computer system is arranged to provide messages to the portable hand held computer providing a control of workflow for the care provider.

6. A system according to claim 5 wherein the computer system is arranged to provide a message to the portable hand held computer of a second care provider in the event that the first care provider provides an indication of an inability to complete a workflow task.

7. A system according to claim 1 wherein there is provided a manual override key which can be engaged with the medical device for overriding the control device.

8. A system according to claim 1 wherein there is provided a series of medical devices with common interface for driving actuation of said operable element and a module separate from the medical devices including a battery, drive member and control device for operating the series of medical devices.

9. A system according to claim 8 wherein the module includes a reader for reading a tag on each of the medical devices and wherein the computer system is arranged to allow operation thereof only in the event that the correct medical device is connected.

10. A system according to claim 1 wherein the RFID tags and the computer system include security protocols.

11. A system according to claim 1 wherein the RFID tags and the control device are programmable and reusable.

12. (canceled)

13. A system according claim 1 wherein the computer system is arranged to prevent operation of the operable element if the medical device is not sterilized.

14. A system according to claim 1 wherein the computer system is arranged to prevent operation of the operable element if it is beyond an expiry date.

15. A system according to claim 1 wherein the computer system is arranged to provide on the portable hand held computer details of allowable use of the medical device.

16. (canceled)

17. A system according to claim 1 wherein the RFID tags and the computer system include protocols for Data integrity.

18. A system according to claim 1 wherein the reader reads multiple RFID tags by a protocol utilizing a windowed access mechanism of a plurality of slots, with a series of transponders contending for a slotted channel in a random access fashion.

19. A system according to claim 1 wherein the medical device comprises one of smart containers, smart clamps, smart valves, smart couplers, smart syringes, smart pipettes, smart bandages and smart catheters.

20. A system according to claim 1 wherein the medical device includes a “tamper-proof” or “breach” indicator.

21. (canceled)

22. A system according to claim 1 wherein the portable hand held computer has at least a part of the electronics thereof juxtaposed with an RFID Reader.

23. A system according to claim 1 wherein the medical device at the POC has an RFID tag juxtaposed with interfacing electronics forming at least part of the control device (perhaps RFID System on a Chip).

24. A system according to claim 1 wherein the control device is arranged to disable operation of the operable element.
